(19) United States
(12) Patent Application Publication
(10) Pub. No.: US 2013/0115271 A1
(43) Pub. Date: May 9, 2013

(54) PREDICTORS OF PHARMACOKINETIC AND PHARMACODYNAMIC DISPOSITION OF CARRIER-MEDIATED AGENTS

(71) Applicants: William C. Zamboni, Chapel Hill, NC (US); Whitney Paige Caron, Carrboro, NC (US)

(72) Inventors: William C. Zamboni, Chapel Hill, NC (US); Whitney Paige Caron, Carrboro, NC (US)

(73) Assignee: THE UNIVERSITY OF NORTH CAROLINA AT CHAPEL HILL, Chapel Hill, NC (US)

(21) Appl. No.: 13/642,299

(22) PCT Filed: Apr. 19, 2011
(86) PCT No.: PCT/US11/33020

§ 371 Date: Jan. 17, 2013

Related U.S. Application Data
(60) Provisional application No. 61/325,698, filed on Apr. 19, 2010.

(51) Int. Cl. C12Q 1/02

(52) U.S. Cl. CPC C12Q 1/02
USPC 424/450; 435/29; 435/7.21; 514/34

(57) ABSTRACT

The invention provides a method of predicting the clearance rate of a carrier-mediated agent and/or the release of an agent from a carrier in a subject comprising measuring the number and/or activity of phagocytic cells and/or the amount and/or activity of opsonins and/or the amount and/or activity of complement within a biological sample obtained from a subject, and predicting the clearance rate of the carrier-mediated agent and/or the release of the agent from the carrier based upon the number and/or activity of the phagocytic cells and/or the amount and/or activity of opsonins and/or the amount and/or activity of complement.

### RELATED APPLICATION INFORMATION

This application claims the benefit of U.S. Provisional Application No. 61/325,698; Filed Apr. 19, 2011, the disclosure of which is incorporated by reference herein in its entirety.

### STATEMENT OF FEDERAL SUPPORT

This invention was supported in part by Grant No. CA119343 from the National Cancer Institute. The United States government has certain rights in this invention.

### FIELD OF THE INVENTION

The present invention concerns methods of predicting the clearance rate of a carrier-mediated agent (for example, nanoparticle, liposome, polymer and conjugated drug formulations) and/or release of the agent from the carrier in a subject and, optionally, determining a dosage of the carrier-mediated agent based on the predicted clearance rate and/or release. The invention also concerns methods of identifying carrier-mediated agents having desired pharmacokinetic and pharmacodynamics disposition.

### BACKGROUND OF THE INVENTION

### Ovarian Cancer and the Role of PLD

Ovarian cancer is a disease of the peritoneal cavity (Bookman, INT. J. GYNECOL. CANCER 15(Supp. 3):212 (2005)). While most women with ovarian cancer initially respond to platinum-based therapy, the majority of women will relapse and become platinum-resistant (Bookman, J. CLIN. ONCOL. 21:149s (2003)). Pegylated-liposomal doxorubicin (PLD) is one of the few treatments approved for refractory ovarian cancer (Id.). However, the use of PLD as second-line treatment of platinum and paclitaxel-refractory ovarian cancer has achieved response rates of only 14% to 20% (Id.). In addition, there is significant variability in the pharmacokinetics (PK) and pharmacodynamics (PD) (especially hand-foot syndrome) associated with PLD. Currently, the significant toxicity associated with hand-foot syndrome can only be addressed by decreasing the dose or stopping PLD therapy which further reduces the response rate to PLD. Thus, there is a need to identify the factors associated with the variability in PK, response and toxicity of PLD in order to improve the response rate and the quality of life for women with ovarian cancer.

### Carrier-Mediated Anticancer Agents

Major advances in the use of carrier vehicles delivering pharmacologic agents and enzymes to sites of disease have occurred in the past 10 years (Park et al., SEMIN. ONCOL. 31:196 (2004); Zamboni, THE ONCOLOGIST 13:248 (2008)). The primary types of carrier-mediated anticancer agents are liposomes, nanoparticles and conjugated agents. Liposomes can be subdivided into stabilized and non-stabilized (conventional) liposomes. Stabilized liposomes can also be subdivided into those that are stabilized by polyethylene glycol (PEG) or a non-PEG substitute such as sphingomyelin. Nanoparticles are subdivided into microspheres, which include polymer micelles, and dendrimers. Conjugate formulations consist of the agent linked to PEG or non-PEG polymers (Zamboni, CLIN. CANCER RES. 11:8230 (2005); Zamboni, THE ONCOLOGIST 13:248 (2008)). The theoretical advantages of liposomal and nanoparticle encapsulated and carrier-mediated agents include increased solubility, prolonged duration of exposure, selective delivery of entrapped agent to the site of action, improved therapeutic index, and potentially overcoming resistance associated with the non-carrier-mediated anticancer agent (Id.). PLD (Doxil®, CAELYX®), liposomal daunorubicin (DAUNOXOME®), liposomal cytarabine (DEPOCYT®), and paclitaxel albumin-bound particles (ABRAXANE®) are the only members of this relatively new class that are FDA approved (Id.). Although these are the only FDA approved carrier-mediated chemotherapeutic agents, >200 other agents are in preclinical and clinical development. In addition, antiangiogenesis agents, antisense oligonucleotides, and enzymes represent rational candidates for liposomal and nanoparticle formulations (Park et al., SEMIN. ONCOL. 31:196 (2004)).

### PK and PD Disposition of Carrier-Mediated Agents

The PK disposition of carrier-mediated agents, such as liposomes, nanoparticles and conjugated agents, is dependent upon the carrier and not the encapsulated agent until the agent is released from the carrier (Langinha et al., BIOCHIM. BIOPHYS. ACTA 1711:25 (2005); Papahadjopoulos et al., PROC. NATL. ACAD. SCI. USA 88:11460 (1991); Zamboni, THE ONCOLOGIST 13:248 (2008)). PK parameters of the liposomes and nanoparticles depend on the physiochemical characteristics of the carrier, such as size, surface charge, membrane lipid packing, steric stabilization, dose and route of administration (Zamboni, CLIN. CANCER RES. 11:8230 (2005)). The agent that remains encapsulated within liposomes or nanoparticles, or linked to a conjugate or polymer is in an inactive form, thus the agent must be released from the carrier to be active (Zamboni, CLIN. CANCER RES. 11:8230 (2005); Zamboni et al., CANCER CHEMOTHER. PHARMACOL. 53:329 (2004); Zamboni, THE ONCOLOGIST 13:248 (2008)). Whether the agent needs to be released outside of the cell in the tumor extracellular fluid (ECF) or within the cell depends on the formulation of the carrier and the mechanism of release (See, e.g., D'Emanuele and Atwood, ADV. DRUG DELIV. RES. 57:2147 (2005); Papahadjopoulos et al., PROC. NATL. ACAD. SCI. USA 88:11460 (1991); Zamboni, CLIN. CANCER RES. 11:8230 (2005); Zamboni, THE ONCOLOGIST 13:248 (2008)). After the agent is released from the carrier, the PK disposition of the agent will be the same as following administration of the non-carrier form of the agent (Id.). Thus, the pharmacology and PK of these agents is complex. The nomenclature used to describe the PK disposition of carrier-mediated agents includes encapsulated or conjugated (agent within or bound to the carrier), released (the active agent released from the carrier), and sum total (carrier-mediated agent plus released agent) (Zamboni, CLIN. CANCER RES. 11:8230 (2005); Zamboni, THE ONCOLOGIST 13:248 (2008)). The released agent has also been called the legacy drug, regular drug, or warhead (Yurkovetskiy et al., MOL. PHARM. 1:375 (2004); Zamboni, CLIN. CANCER RES. 11:8230 (2005); Zamboni, THE ONCOLOGIST 13:248 (2008)). Released agent consists of agent that is protein bound and unbound (or free) agent.

Nanoparticle, liposomal, and conjugated agents are cleared via the reticuloendothelial cell system (RES), also called the mononuclear phagocyte system (MPS), which is located primarily in the liver and spleen (Siedner et al., J. PARENTER. ENTERAL. NUTR. 13:614 (1989)). Non-pegylated or non-stabilized nanoparticles are cleared relatively quickly via the RES. Pegylated or stabilized nanoparticles also are cleared via the RES but at a much slower rate than non-stabilized carriers. Nanoparticles can alter both the tissue distribution and the clearance of agents because the agent takes on the PK characteristics of the carrier (Maeda et al., J. CONTROL. RELEASE 65:271 (2000)). The primary sites of accumulation of conventional liposomes are tumors, liver and spleen as compared with non-liposomal formulations (Id.).

### Factors Affecting RES Function

The primary cells of the RES are monocytes, macrophages, and dendritic cells. Monocytes in blood can be activated by nanoparticle drugs and other foreign antigens. Monocyte activation can result in migration into tissue and interstitial space and differentiation into macrophages which have high phagocytic activity. Dendritic cells in blood can also be activated by nanoparticles and foreign antigens. While in the blood, dendritic cells actively engulf these particles. After phagocytosis, dendritic cells mature into antigen presenting cells and migrate into tissue such as the spleen where they are believed to be directly involved in the stimulation and maturation of T lymphocytes. Several physiologic changes and disease-related alterations in organ function occur with aging and these changes can affect the PK of drugs in older persons (Cusack, AM.J. GERIATR. PHARMACOTHER. 2:274 (2004)). There is an age-related decline in hepatic metabolism and renal drug elimination typically declines with age, commensurate with the fall in creatinine clearance. In addition, fat-soluble agents, such as liposomal drugs, may distribute differently in older people compared with younger people. There is also a well-documented decline in function of the immune system in older people (Arlt and Hewison, AGING CELL 3:209 (2004)). Macrophage, monocyte and dendritic cell function is variably impaired in older patients and in patients with cancer which may alter the clearance of liposomal agents. However, few studies have systematically measured changes in monocyte, macrophage, and dendritic cell number and/or function in the elderly or in patients with cancer.

### Advantages of Phenotypic Probes

The therapeutic index of anticancer agents is small as compared with other non-chemotherapy drugs. In addition, the PK and PD variability of liposomal anticancer agents administered intravenously (IV) is several fold higher as compared with small molecule anticancer agents administered orally or IV. Furthermore, it appears that liposomal, nanoparticle and conjugated agents are all cleared via the RES and have high PK and PD variability. These factors raise serious concerns about the translational development and clinical utility of nanoparticle anticancer agents. Thus, there is a need to identify, reduce and/or compensate for the factors associated with the high PK and PD variability of nanoparticle anticancer agents as methods to improve response. Moreover, the numerous current and future carrier-mediated agents would benefit from methods to address these PK and/or PD issues in order to develop effective anticancer agents.

### SUMMARY OF THE INVENTION

Carrier-mediated anticancer agents include nanoparticles, liposomes, conjugates and polymer carriers. The interpatient variability in the disposition of carrier-mediated agents is significantly greater than the released form of the agent. The present inventors have determined that the high and clinically relevant interpatient variability in PK and PD of carrier-mediated chemotherapy drugs is related to the function of monocytes (MO) and dendritic cells (DC) of the reticuloendothelial system (RES), which serves as the clearance pathway for carrier-mediated agents.

Accordingly, a first aspect of the invention is a method of predicting the clearance rate of a carrier-mediated agent in a subject, the method comprising:

a) measuring the number and/or activity of phagocytic cells in a biological sample obtained from the subject; and

b) predicting the clearance rate of the carrier-mediated agent in the subject based on the number and/or activity of the phagocytic cells in the biological sample.

The invention also provides a method of predicting the release of an agent from a carrier-mediated agent, the method comprising:

a) measuring the activity of phagocytic cells in a biological sample obtained from the subject; and

b) predicting the release of the agent from the carrier-mediated agent in the subject based on the activity of the phagocytic cells in the biological sample.

In representative embodiments, the carrier-mediated agent is a carrier-mediated drug (e.g., pegylated liposomal encapsulated doxorubicin).

In embodiments of the invention, the biological sample is contacted (e.g., incubated) with the carrier-mediated agent or a surrogate (e.g., the “empty” carrier) prior to measuring the activity of phagocytic cells in the biological sample.

Optionally, according to this embodiment, the predicted values are compared with the actual values. For example, the actual clearance rate of the carrier-mediated agent in the subject can be determined and compared with the predicted clearance rate. Likewise, the actual release of the agent from the carrier in the subject can be determined and compared with the predicted release of the agent from the carrier.

In embodiments of the invention, predicting the clearance rate of the carrier-mediated agent and/or predicting the release of the agent from the carrier-mediated agent comprises comparing the number and/or activity of the phagocytic cells within the biological sample to a reference value. Optionally, the reference value is based on the number and/or activity of phagocytic cells within biological samples taken from healthy subjects or from affected subjects.

In embodiments of the invention, the method further comprises obtaining the biological sample from the subject (e.g., prior to the measuring step).

In embodiments of the invention, the sample is a blood sample, plasma sample, serum sample, ascites sample (e.g., malignant ascites), or any combination of the foregoing.

In embodiments of the invention, the subject is a human subject.

In embodiments of the invention, the subject is receiving or will receive chemotherapy. Optionally, the method is carried out prior to two or more cycles of chemotherapy to determine an individualized dosage prior to each cycle. In representative embodiments, the method is carried out prior to every cycle of chemotherapy.

In embodiments of the invention, the carrier-mediated agent comprises a liposome, a nanoparticle, a conjugate and/or a polymer. For example, the carrier-mediated agent can comprise a stabilized liposome, a non-stabilized liposome, a nanosphere, a microsphere, a dendrimer, a quantum dot, a gold nanoshell, a nanocrystal, colloidal gold, a nanoemulsion, an antibody, a viral vector, a virus-like particle, a carbon nanotube, a gold nanoparticle, a silver nanoparticle, a silica nanoparticle, a conjugate, a polymer, or any combination thereof.

In embodiments of the invention, the activity of phagocytic cells is measured by evaluating phagocytosis, respiratory burst activity, chemotaxis, receptor binding, generation of superoxide, generation of nitric oxide, presentation of one or more antigens at the cell surface, or any combination thereof.

In embodiments of the invention, the phagocytic cells comprise monocytes, macrophages, dendritic cells (e.g., myeloid and/or lymphoid DC), granulocytes, mast cells, lymphocytes, or any combination thereof. In embodiments of the invention, the cell is a Peripheral Blood Mononuclear Cell (PBMC).

In embodiments of the invention, the method further comprises determining the amount and/or activity of opsonins in the biological sample.

In embodiments of the invention, the method further comprises determining the amount and/or activity of complement in the biological sample.

In embodiments of the invention, the carrier-mediated agent comprises a detectable label.

As a further aspect, the invention also provides a method of selecting a dosage of a carrier-mediated drug for a subject, the method comprising:

(a) measuring the number and/or activity of phagocytic cells in a biological sample obtained from the subject;

(b) predicting the clearance rate of the carrier-mediated drug in the subject based on the number and/or activity of the phagocytic cells in the biological sample; and

(c) selecting a dosage of the carrier-mediated drug for the subject from the predicted clearance rate.

Optionally, the method further comprises administering the dosage of the carrier-mediated drug to the subject.

In representative embodiments, the carrier-mediated drug is pegylated liposomal encapsulated doxorubicin.

In embodiments of the invention the biological sample is contacted with the carrier-mediated drug or drug surrogate prior to measuring the activity of phagocytic cells in the biological sample.

This aspect of the invention can optionally comprise any of the additional features described herein.

As still another aspect, the invention provides a method of predicting the activity of the reticuloendothelial cell system (RES) in a subject, the method comprising:

(a) measuring the number and/or activity of phagocytic cells in a biological sample obtained from the subject; and

(b) predicting the activity of the RES in the subject based on the number and/or activity of the phagocytic cells in the biological sample.

In embodiments of the invention, the biological sample is contacted with a carrier-mediated agent prior to measuring the number and/or activity of phagocytic cells in the biological sample.

This aspect of the invention can optionally comprise any of the additional features described herein.

As still yet a further aspect, the invention provides a method of identifying a carrier-mediated agent having a desired effect (e.g., a stimulatory effect) on and/or interaction with the RES, the method comprising:

(a) measuring the number and/or activity of phagocytic cells in a biological sample obtained from a subject;

(b) predicting the effect of the carrier-mediated agent on the RES and/or the level of interaction of the carrier-mediated agent with the RES in the subject based on the number and/or activity of the phagocytic cells in the biological sample; and

(c) identifying a carrier-mediated agent with a predicted effect on the RES and/or level of RES interaction in a target range based on the prediction of (b).

In embodiments of the invention, the method is carried out with two or more carrier-mediated agents.

In embodiments of the invention, the biological sample is contacted with the carrier-mediated agent prior to measuring the number and/or activity of phagocytic cells in the biological sample.

This aspect of the invention can optionally comprise any of the additional features described herein.

The foregoing and other aspects of the present invention are explained in greater detail in the drawings and the specification set forth below.

### BRIEF DESCRIPTION OF THE DRAWINGS

FIG. 1 shows the relationship between S-CKD602 dose and area under the concentration versus time curve (AUC) of encapsulated CKD-602 in plasma. Interpatient variability ranged from 20- to 100-fold.

FIG. 2 shows DOXIL® AUC/dose in patients <60 years of age and ≧60 years of age. Individual patient data and mean values are represented by the open circles and solid triangles, respectively.

FIG. 3 shows the relationship between the % decrease in monocytes (MO) at nadir in blood and clearance (CL) of encapsulated CKD-602 in plasma after administration of S-CKD602 in patients.

FIG. 4 shows the relationship between the % decrease in MO at nadir in blood and the release of CKD-602 from S-CKD602 in plasma after administration of S-CKD602 in patients with refractory solid tumors.

FIG. 5 shows the relationship between the RES phenotypic probe measuring MO respiratory burst activity (fluorescence intensity of Reactive Oxygen Species) and CL of encapsulated doxorubicin in 5 patients with ovarian cancer.

FIG. 6 shows the relationship between monocyte count determined from a complete blood count (CBC) prior to DOXIL® administration and DOXIL® CL. The R² was 0.17. Thus, there was no relationship between monocyte count and DOXIL® CL.

FIG. 7 shows the relationship between in vivo monocyte phagocytic activity prior to DOXIL® administration and DOXIL® CL. The R² was 0.97.

FIG. 8A shows the relationship between in vivo monocyte respiratory burst activity using fMLP and DOXIL® clearance.

FIG. 8B shows the relationship between in vivo monocyte respiratory burst activity (without the addition of stimulants) and DOXIL® clearance.

FIG. 9 shows the relationship between in vivo monocyte respiratory burst activity by E. coli prior to DOXIL® administration and DOXIL® CL. The R²=0.01, thus there was no relationship.

FIG. 10 shows the relationship between in vivo monocyte respiratory burst activity by PMA prior to DOXIL® administration and DOXIL® CL. The R²=0.01, thus there was no relationship.

FIG. 11 shows the relationship between monocyte phagocytic activity without the ex vivo addition of DOXIL® and DOXIL® CL. The R² value was 0.53.

FIG. 12 shows the relationship between monocyte phagocytic activity with the ex vivo addition of DOXIL® and DOXIL® CL. The R² value was 0.80.

FIG. 13 shows a standard curve showing the relationship between phenotypic measures of RES function and drug CL.

FIG. 14 shows characterization of 8 different carrier-mediated agents (for example, 8 different carriers or nanoparticle formulations) with two phenotypic probes. The left-hand side of the figure demonstrates how carrier-mediated agent can be selected for cancer treatment based on a relatively low effect on the RES, whereas the right-hand side of the figure shows how carrier-mediated agents with relatively strong effects on the RES can be selected for treatment of immune disorders.

### DETAILED DESCRIPTION

The inter-subject variability in the disposition of carrier-mediated drug agents is significantly greater than non-carrier mediated agents. The present invention is based, in part, on the discovery that inter-subject variability in the pharmacokinetics (PK) and pharmacodynamics (PD) of carrier-mediated drug agents is related to the function of the organs and cells of the reticuloendothelial system [RES], which serves as the clearance pathway for carrier-mediated drug agents, particularly the phagocytic cells (e.g., monocytes, macrophages and/or dendritic cells) of the RES.

The invention can be practiced to predict the clearance rate of a carrier-mediated agent and/or release of the agent from the carrier in a subject. Carrier-mediated agents include carrier-mediated drugs. Furthermore, the methods of the invention can be used to select (e.g., optimize, individualize) the dosage of a carrier-mediated drug agent for a particular subject or cohort of subjects (for example, subjects over a certain age) based on the predicted clearance rate and/or release of the drug from the carrier. As non-limiting examples, the dosage can be selected to achieve a targeted exposure level of the carrier-mediated drug (e.g., area under the concentration versus time curve [AUC] for the encapsulated and/or released drug), to improve drug efficacy (e.g., to select a therapeutically effective amount or exposure of the drug), to reduce toxicity and/or to improve the therapeutic index (i.e., the difference in the exposure thresholds associated with response [lower] and toxicity [higher]).

The invention also contemplates the use of the methods described herein to identify an appropriate animal model for preclinical studies (e.g., toxicology, efficacy and/or pharmacology studies) of a carrier-mediated drug agent, for example, to identify an animal model for studies in support of an application for regulatory approval.

The invention further encompasses use of the methods described herein to screen carrier-mediated agents for pharmacologic, efficacy and/or toxicologic effects, e.g., as part of an in vitro, ex vivo and/or in vivo system. For example, a carrier-mediated agent can be selected based on the desired level of activation of phagocytic cells.

As a further aspect, the invention also contemplates use of the methods of the invention to demonstrate that two or more carrier-mediated agents (e.g., carrier-mediated drugs) have similar or dissimilar properties. For example, the method can be used to demonstrate that a generic carrier-mediated drug is bioequivalent to the innovator product (e.g., induces the same or similar level of activation of the RES system) or to identify a generic carrier-mediated drug that is bioequivalent. As another option, the invention can be practiced to identify carrier-mediated agents (e.g., carrier-mediated drugs) that have improved PK and PD parameters as compared with a known carrier-mediated drug, e.g., to improve therapeutic index.

In representative embodiments, the invention is used to assess the effect of a carrier-mediated agent on the immune system of a subject (e.g., RES). For example, the invention can be practiced to assess the effects of environmental exposure to carrier-mediated drugs and other carrier-mediated agents (e.g., carrier-mediated agents comprising a carrier that is a carbon nanotube, gold nanoparticle, silver nanoparticle, silica nanoparticle, polymer [e.g., PEG and/or PLGA], and the like) on the immune system.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The present invention encompasses any and all possible combinations of the features described herein.

The present invention also contemplates that in some embodiments of the invention, any feature or combination of features set forth herein can be excluded or omitted.

As used herein, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a carrier-mediated agent, time, temperature, enzymatic activity or other biological activity and the like, is meant to encompass variations of 20%, 10%, 5%, 1%, 0.5%, or even 0.1% of the specified amount.

As used herein, the term “consisting essentially of” is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976); see also MPEP §2111.03. Thus, the term “consisting essentially of” as used herein should not be interpreted as equivalent to “comprising.”

As used herein, a “biological sample” may comprise any suitable body fluid, tissue and/or excreta in which phagocytic cells may be present. Suitable body fluids include, but are not limited to, lymph, blood, plasma, serum, urine, semen, saliva, cerebrospinal fluid and/or ascites fluid. Suitable tissues include, but are not limited to, spleen tissue, liver tissue, renal tissue, connective tissue, smooth muscle tissue, cardiac muscle tissue, skeletal muscle tissue, bone marrow tissue, nervous system tissue, epithelial tissue, skin, and/or lymph nodes. Excreta includes feces, urine and/or sweat.

The biological sample can be collected at any suitable time. For example, the biological sample can be collected prior to, during and/or after exposure to a carrier-mediated agent, whether such exposure is intentional or not. For example, the exposure can be an environmental exposure (e.g., in the workplace). As another option, the subject can be administered a carrier-mediated drug and the biological sample can be obtained prior to (e.g., within about 1, 2, 3, 4, 5, 6, 7, 14, 21, 30 or 45 days), during and/or after (e.g., immediately following or within about 1, 2, 3, 4, 5, 6, 7, 14, 21, 30 or 45 days) administration of the carrier-mediated drug. Generally, collection of the biological sample at least one day prior to administration of a carrier-mediated drug is convenient for determining a dosage to be administered based on the predicted clearance rate and/or the predicted release of the drug from the carrier (e.g., at least about 1, 2, 3, 4, 5, 6, 7, 14, 21, 30 or 45 days prior to administration of the carrier-mediated drug).

“Carriers” for agents and drugs according to the present invention include but are not limited to liposomes, nanoparticles, conjugates and polymers (e.g., a polymer nanosphere). As used herein, the terms “carrier-mediated agent,” “carrier-mediated drug” and like terms include, but are not limited to, agents/drugs encapsulated within liposomes or nanoparticles, agents/drugs embedded in liposomes or nanoparticles, agents/drugs attached to the outer surface of liposomes or nanoparticles, and/or agents/drugs that are conjugated to a carrier molecule. Liposomes may optionally be non-stabilized or stabilized, e.g., with polyethylene glycol (PEG) and/or with a non-PEG substitute such as sphingomyelin. Nanoparticles include nanospheres (including polymer nanospheres), microspheres (including polymer micelles), dendrimers, quantum dots, gold nanoshells, nanocrystals, colloidal gold, nanoemulsions, antibodies (e.g., HERCEPTIN®, RITUXAN® or ERBITUX®), viral vectors, virus-like particles, carbon nanotubes, gold nanoparticles, silver nanoparticles, silica nanoparticles, and the like. Exemplary conjugate formulations can comprise PEG, poly(lactic-co-glycolic acid) (PLGA) and/or another polymer as a carrier molecule. By way of example, polymer based carrier-mediated agents can comprise, without limitation, PEG and/or PLGA. In representative embodiments, the polymer based carrier-mediated agent is a PRINT® (Particle Replication in Non-wetting Templates) nanoparticle (see, e.g., Gratton et al., (2008) Pharm. Res. 25:2845-2852).

The carrier-mediated agent can comprise any suitable active agent, including without limitation small molecules, protein or peptide agents (e.g., enzymes, antibodies, antibody fragments), lipid agents, oligonucleotide agents (e.g., antisense oligonucleotides, RNAi), and/or carbohydrate agents.

Likewise, according to the present invention, a carrier-mediated drug can be intended for any therapeutic indication. In embodiments of the invention, the carrier-mediated drug is used to treat cancer patients (e.g., a chemotherapeutic drug). In representative embodiments of the invention, the carrier-mediated drug is an anti-angiogenesis agent.

Exemplary carrier-mediated agents include without limitation PLD (e.g., DOXIL®, CAELYX®), liposomal daunorubicin (e.g., DAUNOXOME®), liposomal cytarabine (e.g., DEPOCYT®), paclitaxel albumin-bound particles (e.g., ABRAXANE®), amphotericin B liposome (AMBISOME®), amphotericin B lipid complex (e.g., ABELCET®) and pegylated liposomal CKD-602, a camptothecin analogue (e.g., S-CKD602).

Table 1 provides a listing of exemplary carrier-mediated chemotherapeutic agents that may be used in conjunction with the present invention.

TABLE 1
Summary of carrier-mediated chemotherapeutic agents
Carrier-mediated chemotherapeutic agents

| Nanosomes | | | | |
|---|---|---|---|---|
| Conventional | Stabilized | | | |
| (Nonstabilized) | Nonpegylated | Pegylated | Nanoparticles | Conjugates and polymers |
| LE-SN38 | Optisomal topotecan | Doxil ® (Caelyx ®)^{a} | Abraxane ® (AB1007) | Xyotax ® (PPX; CT-2103; |
| | (TL1) | | | paclitaxel) |
| Lurtotecan/OSI-211 | Optisomal vincristine | S-CKD602 (AP-30)^{a} | Paclimer ® (paclitaxel) | MER-1001 (CPT) |
| 9NC | Optisomal vinorelbine | IHL-305 (irinotecan) | Tocosol ® paclitaxel | 1T-101 (CPT) |
| Irinotecan | AT1-1123 | Nanoliposomal | APA-thalidomide | DHA-paclitaxel |
| | (PSN-docetaxel) | CPT-11 | | |
| LEP-ETU (paclitaxel) | | | PX-NP (paclitaxel) | PEG-doxorubicin |
| Docetaxel | | | NK-012 (SN38) | PEG-methotrexate |
| Doxorubicin | | | NK 105 (paclitaxel) | PEG-interferon |
| Daunorubicin | | | ANX-514 (docetaxel) | PEG-camptothecin |
| Cytarabine | | | CNF1010 (17-AAG) | ENZ-2208 |
| | | | | PEG-SN38 |
| Topotecan | | | | NKTR-102 |
| | | | | PEG-irinotecan |
| FRL-doxorubicin: | | | | 20-carbonate-CPT |
| vincristine | | | | |
| FRL-daunorubicin: | | | | PL-ara-C |
| cytarabine | | | | |
| FRL-floxuridine: | | | | PL-gemcitabine |
| irinotecan | | | | |
| FRL-cisplatin: | | | | CT-2106 (camptothecin) |
| irinotecan | | | | |
| DACH L-NDDP | | | | AP-5346 DACH-HPMA |
| AR-726 | | | | (oxaliplatin analogue) |
| (oxaliplatin analogue) | | | | |
| MBP-426 | | | | AP-5280 (cisplatin-analogue |
| (oxaliplatin) | | | | HPMA) |

^{a}Doxil ® (Caelyx ®) and S-CKD602 (AP-30) are pegylated-STEALTH ® liposomal agents.
Abbreviations:
AP-5280 is a cisplatin-like analogue bound to copolymer HPMA;
AP-5346: DACH-HPMA, an oxaliplatin analogue bound to copolymer HPMA;
APA, alginate-poly-lysine-alginate microcapsules;
Ara-C, cytosine arabinoside;
CPT, camptothecin;
CT-2106, polyglutamated camptothecin;
DACH-L-NDDP: liposomal DACH platinum, AR-726, an oxaliplatin analogue;
DHA, docosahexaenoic acid;
FRL, fixed ratio liposomes;
NK-012, micellar SN38;
PEG, polyethylene glycol;
PL, phospholipids;
PSN, protein stabilized nanoparticle for liposomes;
PX-NP = paclitaxel entrapped in cetyl alcohol/polysorbate nanoparticles.

In embodiments of the invention, the carrier-mediated agent is intended to be delivered orally, intravenously, intraperitoneally, topically, via the lymphatic system, via intratumoral injection and/or via a sustained release implant or depot.

Carrier-mediated agents of the present invention may comprise (e.g., be conjugated to) a detectable tag or detectable label. Such a tag can be any suitable tag that allows for detection of the carrier-mediated agent and includes, but is not limited to, any composition or label detectable by spectroscopic, photochemical, biochemical, immunochemical, radiographic, electrical, optical or chemical means. Useful labels include without limitation biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. In embodiments of the invention, the carrier-mediated agent is “double” tagged in that both the carrier and the agent comprise (e.g., are conjugated to) a detectable tag, optionally different detectable tags.

As used herein, the term “cancer” refers to any benign or malignant abnormal growth of cells. Examples include, without limitation, breast cancer, prostate cancer, lymphoma, skin cancer, pancreatic cancer, colon cancer, melanoma, ovarian cancer (including epithelial ovarian cancer and/or recurrent and/or platinum-resistant or platinum-refractory ovarian cancer), brain cancer (e.g., primary brain carcinoma, glioma, and glioblastoma multiforme), head or neck cancer, liver cancer, bladder cancer, lung cancer (e.g., non-small cell lung cancer), Wilms' tumor, cervical cancer, testicular cancer, stomach cancer, prostate cancer, genitourinary cancer, thyroid cancer, esophageal cancer, myeloma (e.g., multiple myeloma), adrenal cancer (e.g., adrenal cortex cancer), renal cell cancer, endometrial cancer, malignant carcinoid cancer, choriocarcinoma, mycosis fungoides, malignant hypercalcemia, leukemia (e.g., acute lymphocytic leukemia, chronic lymphocytic leukemia, acute myelogenous leukemia, chronic myelogenous leukemia, chronic granulocytic leukemia, acute granulocytic leukemia, hairy cell leukemia), neuroblastoma, polycythemia vera, essential thrombocytosis, lymphoma (e.g., Hodgkin's disease, non-Hodgkin's lymphoma), sarcoma (e.g., soft-tissue sarcoma, osteogenic sarcoma, rhabdomyosarcoma, Kaposi's sarcoma), primary macroglobulinemia, and retinoblastoma. In some embodiments, the cancer is a tumor-forming cancer.

The term “clearance rate” or “drug clearance rate” and similar terms as used herein refer to the rate at which a carrier-mediated agent or drug, respectively, is cleared or taken up by the RES. The term “clearance rate” encompasses “drug clearance rate” and is generally used in the description of the present invention unless drug clearance rate is specifically intended. Organs and cells of the RES responsible for clearance of carrier-mediated agents and drugs include, without limitation, liver, spleen, lung, bone marrow and/or Peripheral Blood Mononuclear Cells (PBMC). With respect to small molecules (not carrier-mediated), the “clearance rate” or “drug clearance rate” refers to the rate at which the small molecule agent or drug is taken up and enzymatically metabolized or excreted by the liver and filtered or excreted by the kidney.

As used herein, “predicting the clearance rate” or “predicting the drug clearance rate” refers to estimating, predicting, determining (and like terms) the absolute or relative clearance rate or drug clearance rate, respectively (as defined above). The term “predicting the clearance rate” encompasses “predicting the drug clearance rate” and is generally used in the description of the present invention unless predicting the drug clearance rate is specifically intended. In particular embodiments, the clearance rate or drug clearance rate is predicted for a particular compartment, e.g., blood, plasma, serum, lymph and/or peritoneal ascites. In other embodiments, a whole body clearance rate or whole body drug clearance rate (i.e., systemic) is predicted.

According to the present invention, clearance rate can be predicted “based on” any of the phenotypic probes discussed herein (e.g., number and/or activity of phagocytic cells, the amount and/or activity of opsonins and/or the amount and/or activity of complement in the biological sample). The correlation between the phenotypic probe and clearance rate can be positive or negative, and can further be linear or curvilinear. Clearance rates can be predicted using any suitable method, for example, using a reference value (discussed in more detail herein). For example, a reference value can be determined using a standard curve or equation defining a relationship between clearance rate of the carrier-associated agent and number and/or activity of phagocytic cells, amount and/or activity of opsonins and/or amount and/or activity of complement in the sample. To illustrate, standard curves can be generated based on actual measurements in a population, which can optionally be matched for species, gender, age and/or race and the like with the test subject.

As used herein, the term “phagocytic cell” includes but is not limited to monocytes, granulocytes (including neutrophils, basophils and/or eosinophils), macrophages, dendritic cells (including myeloid dendritic cells and lymphoid dendritic cells), mast cells or lymphocytes, or subpopulations of any of the foregoing cells, or any combination of the foregoing. In representative embodiments, the phagocytic cells comprise, consist essentially of, or consist of monocytes, macrophages, dendritic cells, granulocytes (including neutrophils, basophils and/or eosinophils), mast cells or lymphocytes (e.g., B cells and/or T cells), subpopulations of any of the foregoing cells, or any combination of the foregoing. In representative embodiments, the phagocytic cells, comprise, consist essentially of, or consist of a cell of the RES, including but not limited to dendritic cells, monocytes or macrophages, subpopulations of any of the foregoing cells, or any combination of the foregoing. In embodiments of the invention, the phagocytic cells, comprise, consist essentially of, or consist of a PBMC, including but not limited to lymphocytes, monocytes or macrophages, or any combination thereof.

The term “activity of phagocytic cells” refers to any suitable activity of phagocytic cells, including, but not limited to, chemotaxis, receptor binding, phagocytosis, oxygen consumption (respiratory burst), generation of superoxide, generation of nitric oxide, or presentation of one or more antigens at the cell surface, or any combination of the foregoing. Methods for determining these and other activities of phagocytic cells are known in the art. For example, the PHAGOTEST® and PHAGOBURST® kits are commercially available from Orpegen Pharma (San Diego, Calif.) for determining phagocytic activity and respiratory burst activity, respectively. The respiratory burst assay can be carried using any suitable reagent to achieve phagocytic cell stimulation. In particular embodiments, cell stimulation is achieved with E. coli, PMA, fMLP and/or a carrier-mediated agent.

As used herein, the term “activity of the RES” refers to measuring the activity of phagocytic cells, or subpopulations thereof, of the RES. For example, the activity of monocytes, macrophages, dendritic cells (e.g., myeloid dendritic cells and lymphoid dendritic cells) and/or PMBC, including subpopulations thereof, can be measured.

A “phenotypic probe” as used herein is a phenotype, biological activity, test or agent that serves as a marker or indicator of the PK and/or PD disposition of an agent, for example, that can be used to predict clearance rate, predict release of the agent from the carrier, individualize the dose of the carrier-mediated drug as a method to improve response and/or therapeutic index. Phenotypic probes according to the present invention include, without limitation, the number of phagocytic cells, phagocytic cell activity (e.g., phagocytosic activity and/or respiratory burst activity), the amount and/or activity of opsonins and/or the amount and/or activity of complement.

The term “release of the agent from the carrier” or “release of the drug from the carrier” and similar terms refer to the disassociation of the agent/drug from the carrier-mediated agent or carrier-mediated drug to generate the released agent or drug, respectively. The term “release of the agent from the carrier” encompasses “release of the drug from the carrier” and is generally used in the description of the present invention unless release of a drug from a carrier is specifically intended. In particular embodiments, “release of the agent from the carrier” or “release of the drug from the carrier” and similar terms refer to the release rate of the agent or drug from the carrier-mediated agent. Release of the agent from the carrier is a PK parameter, and actual release can be determined using methods known in the art. For example, the ratio of drug release can be calculated as the ratio [AUC of released agent]/[AUC of carrier-mediated agent]. Further, one exemplary method of calculating the actual release rate of the agent from the carrier is by compartmental modeling of the concentration versus time profile of the carrier-mediated agent and released agent.

“Released agent” or “released drug” as used herein includes agent/drug that is protein bound (e.g., to blood proteins) and unbound (or “free”).

As used herein, “predicting the release of the agent from the carrier” or “predicting the release of the drug from the carrier” refers to estimating, predicting, determining (and like terms) the absolute or relative amount or rate of release of the agent or drug from the carrier, respectively (as defined above). The term “predicting the release of the agent from the carrier” encompasses “predicting the release of the drug from the carrier” and is generally used in the description of the present invention unless predicting the release of a drug is specifically intended. In particular embodiments, the release of the agent or drug rate is predicted for a particular compartment, e.g., blood, plasma, serum, lymph and/or peritoneal ascites. In other embodiments, a whole body release of the agent from the carrier (i.e., systemic) is predicted.

According to the present invention, release of the agent from the carrier can be predicted “based on” any of the phenotypic probes discussed herein (e.g., number and/or activity of phagocytic cells, the amount and/or activity of opsonins and/or the amount and/or activity of complement in the biological sample). The correlation between the phenotypic probe and release of the agent from the carrier can be positive or negative, and can further be linear or curvilinear. Release of the agent from the carrier can be predicted using any suitable method, for example, using a reference value (discussed in more detail herein). For example, a reference value can be determined using a standard curve or equation defining a relationship between release of the drug from the carrier and number and/or activity of phagocytic cells, amount and/or activity of opsonins and/or amount and/or activity of complement in the sample. To illustrate, standard curves can be generated based on actual measurements in a population, which can optionally be matched for species, gender, age and/or race and the like with the test subject.

As used herein, the terms “side effects” and “toxicity” or similar terms refer to any adverse effect in the subject associated with administration or exposure to a drug or other agent. Such side effects/toxicities of drugs (e.g., chemotherapeutic drugs) include without limitation: fatigue, nausea, neurotoxicity (e.g., neuropathy, loss of hearing, tinnitus, vertigo, loss of cognitive function [chemobrain]), renal toxicity, liver toxicity, cardiac toxicity, loss of skeletal muscle mass and/or function, rash, mouth sores, constipation, diarrhea, alopecia, bone loss, bone marrow impairment (e.g., neutropenia, anemia, thrombocytopenia and/or leucopenia), difficulty breathing, high or low blood pressure, hyperglycemia or hypoglycemia, increased risk of cancer (e.g., increased risk of a secondary cancer), impaired sense of smell and/or taste, Palmar-Plantar Erythrodyesthesia (PPE; “hand-foot syndrome”), or any combination of the foregoing.

A “subject” according to the present invention includes both human subjects for medical purposes and animal subjects for veterinary and drug screening and development purposes (e.g., animal models). Suitable subjects include both avians and mammals, and can be males and/or females. The term “avian” as used herein includes, but is not limited to, chickens, ducks, geese, quail, turkeys, pheasant, parrots, parakeets, and the like. The term “mammal” as used herein includes, but is not limited to, humans, non-human primates (e.g., monkeys, baboons, chimps), cattle, sheep, goats, pigs, horses, cats, dogs, rabbits, rodents (e.g., mice, rats, hamsters), etc. Human subjects include neonates, infants, juveniles, adults and aged subjects (e.g., subjects at least about 55, 60, 65, 70, 75, 80 years or older).

In embodiments of the invention, the subject is immunocompromised (e.g., a subject that has had or is undergoing chemotherapy and/or radiation therapy, a subject with HIV/AIDs, etc.).

In embodiments of the invention, the subject has an impairment in the RES system, e.g., a subject with reduced spleen function (e.g., following splenectomy), reduced liver function and/or reduced number of lymph nodes (e.g., following lymph node removal).

Subjects include healthy humans and animals as well as humans and animals (e.g., animal models) affected by any diseases or disorder. Such diseases/disorders, include, but are not limited to, cancer, muscular dystrophy (including Duchenne or Becker muscular dystrophy), hemophilia A, hemophilia B, multiple sclerosis, amyotrophic lateral sclerosis, diabetes mellitus, Gaucher's disease, Fabry disease, Pompe disease, arthritis, muscle wasting, heart disease (including congenital heart failure or peripheral artery disease), intimal hyperplasia, neurological disorders (including epilepsy), Huntington's disease, Parkinson's disease or Alzheimer's disease, autoimmune diseases, cystic fibrosis, thalassemia, Hurler's disease, Krabbe's disease, phenylketonuria, Batten's disease, spinal cerebral ataxia, LDL receptor deficiency, hyperammonemia, anemia and other blood disorders, arthritis, retinal degenerative disorders (including macular degeneration), glycogen storage diseases and other metabolic defects, diseases of solid organs (including, brain, liver, kidney, spleen and heart) and adenosine deaminase deficiency, and any combination of the foregoing.

Those skilled in the art will appreciate that some methods of the invention use a “surrogate” or “drug surrogate,” a molecule that models the behavior of the carrier-mediated agent or carrier-mediated drug, respectively. The surrogate or drug surrogate can be any substance that interacts with (and optionally stimulates) the phagocytic cells and produces the same or similar effects as the carrier-mediated agent or is otherwise reflective of the effect of the carrier-mediated agent or carrier-mediated drug on the phagocytic cells. In non-limiting representative embodiments, the surrogate can comprise, consist essentially of, or consist of a component of the carrier-mediated agent, e.g., the “empty” carrier itself. In embodiments of the invention, the surrogate or drug surrogate is N-formyl-Met-Leu-Phe (fMLP), phorbol 12-myristate 13-acetate (PMA) and/or E. coli (e.g., opsonized).

A “therapeutically effective” amount as used herein is an amount that provides some improvement or benefit to the subject. Alternatively stated, a “therapeutically effective” amount is an amount that provides some alleviation, mitigation, or decrease in at least one clinical symptom in the subject (e.g., in the case of cancer, reduction in tumor burden, prevention of further tumor growth, prevention of metastasis, or increase in survival time). Those skilled in the art will appreciate that the therapeutic effects need not be complete or curative, as long as some benefit is provided to the subject.

One aspect of the present invention is a method of predicting the clearance rate of a carrier-mediated agent, such as a carrier-mediated drug. In embodiments of the invention, the method comprises measuring the number and/or activity of phagocytic cells in a biological sample from a subject, where the number and/or activity of the phagocytic cells in the biological sample correlates with the clearance rate of the carrier-mediated agent. Optionally, the method further comprises comparing the phagocytic cell number and/or activity with a reference value to predict the clearance rate of the carrier-mediated agent.

In addition, the present invention provides a method of predicting the release (e.g., release rate) of an agent from a carrier-mediated agent, such as a carrier-mediated drug. In embodiments of the invention, the method comprises measuring the number and/or activity of phagocytic cells in a biological sample from a subject, where the number and/or activity of the phagocytic cells in the biological sample correlates with the release (e.g., release rate) of the agent from the carrier. Optionally, the method further comprises comparing the phagocytic cell number and/or activity with a reference value to predict the release of the agent from the carrier.

In representative embodiments, the methods of the present invention comprise obtaining a biological sample from a subject, measuring the number and/or activity of phagocytic cells in the biological sample, and comparing the phagocytic cell number and/or activity with a reference value to predict the clearance rate of a carrier-mediated agent and/or the release of the agent from the carrier in the subject.

In particular embodiments, the methods of the invention further comprise contacting (e.g., incubating for a period of time) the biological sample with the carrier-mediated agent or a surrogate for the carrier-mediated agent prior to measuring the number and/or activity of phagocytic cells in the biological sample.

Embodiments of the invention provide a method of predicting the clearance rate of a carrier-mediated agent (e.g., a carrier-mediated drug) and/or the release of the agent from the carrier, wherein the method comprises measuring the amount and/or activity of opsonins in a biological sample from a subject, where the amount and/or activity of opsonins in the biological sample correlates with the clearance rate of the carrier-mediated agent and/or the release of the agent from the carrier. Optionally, the method further comprises comparing the amount and/or activity of opsonins with a reference value to predict the clearance rate of the carrier-mediated agent and/or the release of the agent from the carrier.

Opsonins bind or “coat” the surface of foreign particles, cells, viruses, and the like and make them more susceptible to phagocytosis. Opsonins include but are not limited to: antibodies (e.g., IgG and IgM), components of the complement system (e.g., C3b, C4b and C3b), mannose-binding lectin, and any combination thereof. The amount and/or activity of one or more opsonins in any combination can be determined in practicing the methods of the invention.

In representative embodiments, the methods of the present invention comprise obtaining a biological sample from a subject, measuring the amount and/or activity of opsonins in the biological sample, and comparing the amount and/or activity of opsonins with a reference value to predict the clearance rate of a carrier-mediated agent in the subject and/or the release of the agent from the carrier. In embodiments of the invention, the method comprises determining the ability of opsonins in the biological sample to bind to the carrier-mediated agent, e.g., by combining the biological sample with the carrier-mediated agent for a time sufficient for the opsonins to bind the carrier-mediated agent and determining the amount of opsonins bound to the carrier-mediated agent.

According to exemplary embodiments of the invention, there is a positive correlation (e.g., linear or curvilinear) between the amount of opsonins and/or the activity of opsonins and/or the amount of opsonins bound to the carrier-mediated agent and the clearance of the carrier-mediated agent in the subject and/or the release of the agent from the carrier.

In particular embodiments, the methods of the invention further comprise contacting (e.g., incubating for a period of time) the biological sample with the carrier-mediated agent or surrogate for the carrier-mediated agent prior to measuring the amount and/or activity of opsonins in the biological sample.

In a further exemplary embodiment, the invention provides a method of predicting the clearance rate of a carrier-mediated agent (e.g., a carrier-mediated drug) and/or the release of the agent from the carrier, wherein the method comprises measuring the amount and/or activity of complement in a biological sample from a subject, where the amount and/or activity of complement in the biological sample correlates with the clearance rate of the carrier-mediated agent and/or the release of the agent from the carrier. Optionally, the method further comprises comparing the amount and/or activity of complement with a reference value to predict the clearance rate of the carrier-mediated agent and/or the release of the agent from the carrier.

The complement system is part of the innate immune system and comprises more than 25 small proteins and peptides and includes serum proteins, serosal proteins, and cell membranes. These proteins are generally synthesized by the liver and circulate in the blood as inactive precursors. The complement system has a number of functions including opsonization of foreign antigens, thereby enhancing phagocytosis. The components of the complement system include without limitation: C1q, C1r, C1s, C2, C2a, C2b, C3, C3a, C3b, C4, C4a, C4b, C5, C5a, C5b, C6, C7, C8, C9n, Factor B, Factor Ba, Factor Bb, Factor D, and/or Properdin. The amount and/or activity of one or more components of the complement system in any combination can be determined in practicing the methods of the invention.

In representative embodiments, the methods of the present invention comprise obtaining a biological sample from a subject, measuring the amount and/or activity of complement in the biological sample, and comparing the amount and/or activity of complement with a reference value to predict the clearance rate of a carrier-mediated agent in the subject and/or the release of the agent from the carrier. In embodiments of the invention, the method comprises determining the ability of complement in the biological sample to bind to the carrier-mediated agent, e.g., by combining the biological sample with the carrier-mediated agent for a time sufficient for the complement to bind the carrier-mediated agent and determining the amount of complement bound to the carrier-mediated agent.

According to exemplary embodiments of the invention, there is a positive correlation (e.g., linear and/or curvilinear) between the amount of complement and/or the activity of complement and/or the amount of complement bound to the carrier-mediated agent and the clearance of the carrier-mediated agent and/or the release of the agent from the carrier in the subject.

In particular embodiments, the methods of the invention further comprise contacting (e.g., incubating for a period of time) the biological sample with the carrier-mediated agent or a surrogate for the carrier-mediated agent prior to measuring the amount and/or activity of complement in the biological sample.

Optionally, in the practice of the present invention, phagocytic activity in the biological sample is determined as well as (i) the amount and/or activity of opsonins; (ii) the amount and/or activity of complement; or (iii) both (i) and (ii).

When used to determine dosages of a carrier-mediated drug, the methods of the invention can be practiced once to determine the dosage for a course of treatment, can be practiced periodically, or can be used prior to every treatment to determine an individualized dosage to administer. For example, in the context of chemotherapy, the inventors have discovered that the chemotherapy itself impacts the number and/or activity of phagocytic cells, thereby changing the clearance rate of the carrier-mediated chemotherapeutic agent and/or release of the agent from the carrier-mediated agent over the course of multiple cycles of chemotherapy. Thus, it may be advantageous to obtain a biological sample from the subject prior to every round, every other round, every three rounds, etc. of chemotherapy so that the dosage can be adjusted in accordance with changes in the predicted clearance rate and/or predicted release of the agent from the carrier.

In particular embodiments, the present invention is used to predict clearance rate, to predict release of the agent from the carrier and/or to select a dosage of a carrier-mediated drug to treat a subject with cancer. For example, the method can be used to predict clearance rate, to predict release of the agent from the carrier and/or select a dosage of PLD to treat a patient with ovarian cancer (including epithelial ovarian cancer and/or recurrent and/or platinum-resistant or platinum refractory ovarian cancer), breast cancer, multiple myeloma and/or Kaposi's sarcoma. Optionally, the dosage of PLD is selected to improve the therapeutic efficacy, to reduce Palmar-Plantar Erythrodyesthesia (PPE; “hand-foot syndrome”) and/or to enhance the therapeutic index in a cancer patient, including but not limited to a patient with ovarian cancer, breast cancer, multiple myeloma and/or Kaposi's sarcoma.

The present invention also encompasses methods of predicting the activity of the RES comprising measuring the number and/or activity of phagocytic cells in a subject or in a sample from the subject. In a representative embodiment, the method comprises, (a) measuring the number and/or activity of phagocytic cells in a biological sample obtained from the subject; and (b) predicting the activity of the RES in the subject based on the number and/or activity of the phagocytic cells in the biological sample. In representative embodiments, the number and/or activity of one or more subpopulations of phagocytic cells is measured. For example, the phagocytic cells can be cells of the RES, e.g., monocytes, macrophages and/or dendritic cells (including myeloid and/or lymphoid DC).

Optionally, the methods of predicting RES activity can comprise measuring the amount and/or activity of opsonins and/or complement in the biological sample, which can be carried out with or without measuring the activity of phagocytic cells.

Further, in predicting RES activity, the biological sample can be contacted (e.g., incubated for a period of time) with a carrier-mediated agent or surrogate for the carrier-mediated agent prior to measuring the activity of phagocytic cells and/or the amount and/or the activity of opsonins and/or complement in the biological sample.

This aspect of the invention can advantageously be used for in vitro screening to characterize carrier-mediated drugs or other carrier-mediated agents based on interactions with the immune system (e.g., the RES), and to identify those carrier-mediated drugs or other carrier-mediated agents with desirable effects (e.g., stimulation) on the immune system (e.g., the RES). For example, the inventive methods can be used to evaluate how a carrier-mediated agent or set of carrier-mediated agents stimulates and/or interacts with the immune system (e.g., the RES). As one non-limiting illustration, in representative embodiments, the invention provides a method of identifying a carrier-mediated agent having a desired effect on and/or interaction with the immune system (e.g., the RES), the method comprising: (a) measuring the number and/or activity of phagocytic cells and/or the amount and/or the activity of opsonins and/or complement in a biological sample obtained from a subject; (b) predicting the effect (e.g., a stimulatory effect) of the carrier-mediated agent on and/or the level of interaction of the carrier-mediated agent with the immune system in the subject based on the measurement of (a); and (c) identifying a carrier-mediated agent with a predicted effect on the immune system and/or level of immune system interaction in a desired or target range based on the prediction of (b). The method can be carried out with a plurality of carrier-mediated agents (e.g., the method is carried out with two, three, four, five, six, ten, twenty or more), and optionally the results obtained with each carrier-mediated agent compared to guide the selection of one or more of the carrier-mediated agents for further evaluation.

Further, the invention provides a method of selecting a suitable carrier-mediated agent for a subject, based on the predicted effect and/or level of interaction with the RES/immune system. In representative embodiments, the method comprises: (a) measuring the number and/or activity of phagocytic cells and/or the amount and/or the activity of opsonins and/or complement in a biological sample obtained from a subject; (b) predicting the effect (e.g., a stimulatory effect) of the carrier-mediated agent on and/or the level of interaction of the carrier-mediated agent with the immune system in the subject based on the measurement of (a); and (c) selecting a carrier-mediated agent for the subject with a predicted effect on the immune system and/or level of immune system interaction in a desired or target range based on the prediction of (b). The method can be carried out with a plurality of carrier-mediated agents (e.g., the method is carried out with two, three, four, five, six, ten, twenty or more), and optionally the results obtained with each carrier-mediated agent compared to guide the selection of one or more of the carrier-mediated agents for further evaluation.

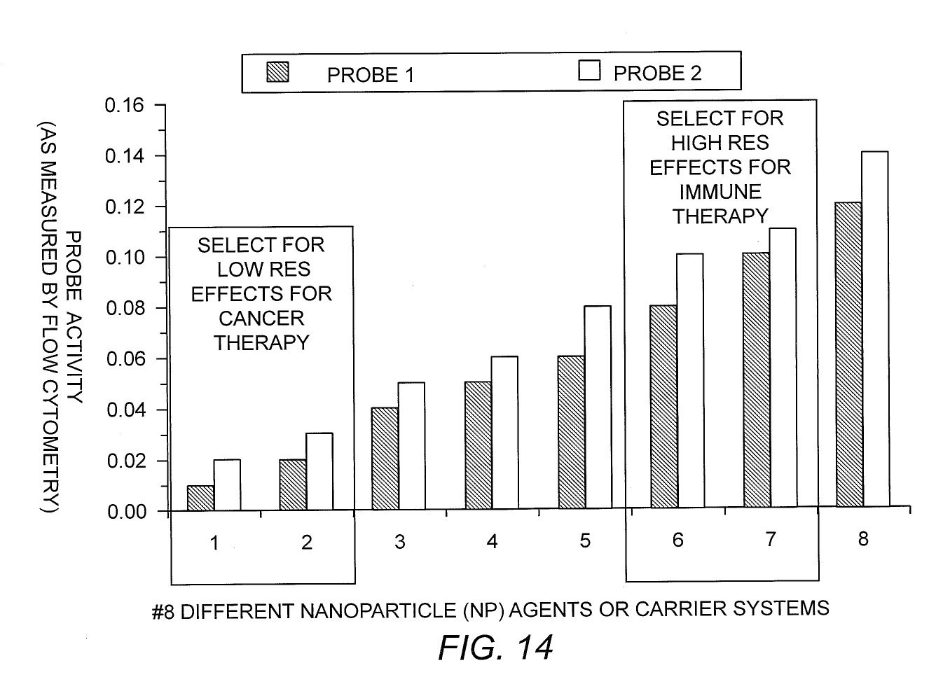

In embodiments of the invention, it is desirable to select chemotherapeutic agents with relatively low levels of stimulation of the immune system (e.g., RES), e.g., so that there is less interaction between the chemotherapeutic drug and the cells of the RES, which may reduce toxicity to the RES and immune system. In contrast, in representative embodiments, carrier-mediated agents that have a relatively high level of stimulation for the immune system (e.g., the RES) are desirable (see FIG. 14), e.g., to treat an immune system disorder a drug that interacts strongly with, and is targeted to, the cells of the RES/immune system may be desirable.

In representative embodiments, the methods of the invention can be practiced to screen for an optimal carrier-mediated agent. For example, variations on a particular carrier can be screened to identify one or more with desired effects on the immune system (e.g., the RES). Alternatively, different carriers can be screened to identify those with the desired interaction with the immune system (e.g., the RES).

In addition, the screening methods can be practiced to show that two carrier-mediated drugs are bioequivalent (e.g., have similar effects on the immune system [e.g., RES]).

The inventive methods of screening carrier-mediated agents for desirable PK and PD characteristics are amenable to high throughput screening, and can be practiced as a manual, semi-automated or fully automated method. The screening methods of the invention can be more cost effective than traditional methods relying on in vivo testing in an animal model. Further, the in vitro methods of the invention can be used to identify carrier-mediated agents for further evaluation in vivo. For example, one or more promising carrier-mediated agents (e.g., carrier-mediated drugs) can be selected based on the methods of the invention and these select carrier-mediated agents can then be evaluated in vivo.

The invention also contemplates the use of the methods described herein to identify an appropriate animal model for preclinical studies (e.g., toxicology, efficacy and/or pharmacology studies) of a carrier-mediated drug agent, for example, to identify an animal model for studies in support of an application for regulatory approval. To illustrate, biological samples can be obtained from one or more candidate animal models (e.g., mouse, rat, hamster, rabbit, dog, pig, monkey, baboon, and the like). The biological samples can be contacted with the carrier-mediated agent of interest (or a surrogate thereof), and then the effects on phagocytic cell number and/or activity and/or the amount and/or activity of opsonins and/or the amount and/or activity of complement can be determined in the biological sample. The effect of the carrier-mediated agent on these aspects of the immune system in the animal model can be compared with the effects observed in human subjects. An animal model can be chosen (e.g., for pre-clinical testing) that has a similar RES response to the carrier-mediated agent as humans.

The invention also encompasses methods of comparing two or more agents, e.g., to determine whether they are similar or different in their effect on the immune system (e.g., RES system). For example, the method can be used to demonstrate that a generic carrier-mediated drug is bioequivalent to the innovator product, e.g., induces the same or similar level of activation of the RES system as determined by phagocytic cell number and/or activity and/or induces the same or similar level and/or activity of opsonins and/or complement.

Alternatively, as another option, the invention can be practiced to identify carrier-mediated agents (e.g., carrier-mediated drugs) that have improved PK and PD parameters as compared with a known carrier-mediated drug, e.g., to improve therapeutic index. For example, variations on a particular carrier formulation can be evaluated to identify a carrier with an optimized clearance profile. As another option, different classes of carriers can be assessed to identify a carrier-mediated agent with a desired level of interaction with the immune system (e.g., the RES).

For example, the release of the agent from the carrier is a PK parameter that can be used to evaluate the stability of a carrier-mediated agent in vitro, ex vivo and in vivo. Carrier-mediated agents can be selected, and optionally compared, based on the release of the agent from the carrier to achieve a desired PK profile and/or stability. Further, release of the agent (e.g., a drug) from a carrier-mediated agent may affect the efficacy and/or toxicity of the agent. A relatively fast or slow release of the agent from the carrier may be correlated (e.g., positively correlated) with improved efficacy depending on the nature of the agent. Likewise, a relatively fast or slow release of the agent from the carrier may be correlated (e.g., positively correlated) with increased side effects/toxicity depending on the nature of the agent. For any particular agent, a reference population can be evaluated to determine the desired or target release of the agent from the carrier, and carriers can be evaluated, and optionally compared, based on the predicted release of the agent from the carrier according to the methods of the invention.

In representative embodiments of the methods of the invention, measuring the activity of phagocytic cells in the biological sample comprises: (i) measuring phagocytic activity and/or respiratory burst activity of monocytes in a biological sample from the subject; (ii) measuring phagocytic activity and/or respiratory burst activity of macrophages in a biological sample from the subject; (iii) measuring phagocytic activity and/or respiratory burst activity of dendritic cells in a biological sample from the subject; (iv) measuring phagocytic activity and/or respiratory burst activity of granulocytes (e.g., neutrophils, basophils and/or eosinophils) in a biological sample from the subject; (v) measuring phagocytic activity and/or respiratory burst activity of mast cells in a biological sample from the subject; (vi) measuring phagocytic activity and/or respiratory burst activity of lymphocytes in a biological sample from the subject; or (vii) any combination of (i) to (vi).

In representative embodiments of the methods of the invention, determining the number of phagocytic cells in the biological sample comprises: (i) determining the number of monocytes in a biological sample from the subject; (ii) determining the number of macrophages in a biological sample from the subject; (iii) determining the number of dendritic cells in a biological sample from the subject; (iv) determining the number of granulocytes (e.g., neutrophils, basophils and/or eosinophils) in a biological sample from the subject; (v) determining the number of mast cells in a biological sample from the subject; (vi) determining the number of lymphocytes in a biological sample from the subject; or (vii) any combination of (i) to (vi).

According to the present invention, one or more activities of phagocytic cells and/or the number of phagocytic cells, the amount and/or activity of opsonins and/or the amount and/or activity of complement (e.g., the phenotypic probe) are measured (e.g. determined) and used to predict the clearance rate of the carrier-mediated agent and/or to predict the release of the agent from the carrier in a subject. The predicted clearance rate and/or release of the agent from the carrier can be based on quantitative, semi-quantitative and/or qualitative measurements. Quantitative methods can be used to determine a relative or absolute clearance rate and/or release of the agent from the carrier.

In semi-quantitative and quantitative methods, a reference value can be determined by any means known in the art, and is optionally a predetermined standard.

In particular embodiments, the reference value is based on known values derived from healthy and/or affected subject populations (e.g., a standard curve). The relationship between the phenotypic probe and the predicted clearance rate can be positive or negative, and can further be linear or curvilinear. For example, there can be a positive and linear correlation between the activity of phagocytic cells and predicted clearance rate of a carrier-mediated agent and/or release of the agent from the carrier. In representative embodiments, the subject can be compared with an unselected population and/or with a population of healthy (i.e. unaffected) subjects and/or a population of affected subjects. By “affected” subject, is meant a subject with the same or similar condition. In embodiments of the invention, the subject is compared with an age-matched population as there is a trend towards reduced hepatic metabolism and reduced renal drug elimination with age (Cusak, AM. J. GERIATR. PHARMACOTHER. 2:274 (2004)), as well as a documented decline in the overall function of the immune system in older individuals (Arlt and Hewison, AGING CELL 3:209 (2004)). The subject can further be matched with a gender-matched population, and for women can optionally be matched for menopausal status, as both of these factors are believed to alter immune function.

In alternative embodiments, the reference value is predetermined in the sense that it is fixed, for example, based on previous experience with the assay and/or a population of subjects. Alternatively, the term “predetermined standard” can also indicate that the method of arriving at the reference value is predetermined or fixed even if the particular value varies among assays or may even be determined for every assay run.

The reference value can be tied to any desired parameter or combination of parameters, e.g., a clearance rate that is associated with a desired level of drug exposure (e.g., AUC of the encapsulated and/or released drug), a release rate of the drug from the carrier that is associated with improved efficacy and/or reduced toxicity, half-life, level of drug efficacy, level of adverse side effects and/or therapeutic index.

Drug dosages can be selected based on the predicted clearance rate of a carrier-mediated drug (e.g., a dosage for the subject). In embodiments of the invention, the selected dosage of the carrier-mediated drug has a positive correlation with the predicted clearance rate (e.g., there is a positive and linear correlation between the selected dosage of a liposome encapsulated drug such as PLD and clearance rate as predicted by respiratory burst and/or phagocytic activity [e.g., monocyte phagocytic activity]). In other embodiments, the predicted clearance rate has a negative correlation with the selected dosage. In embodiments of the invention, these results are extrapolated to any drug being delivered with the same (or structurally similar) carrier or same class of carrier (e.g., liposomes).

Any method known in the art such as a standard curve or equation can be used to adjust the dosage of a carrier-mediated drug based on the relationship with the clearance rate. In representative embodiments, the selected dosage varies linearly and in a positive fashion with the predicted clearance rate, e.g., if the predicted clearance rate is twice the reference value, then the dosage of the drug is increased two-fold to get a target drug exposure in the subject. The dosage of carrier-mediated drugs with non-linear clearance can also be adjusted using these methods in a similar fashion once the relationship between dose and clearance rate is defined (e.g., using a standard curve or equation).

In representative embodiments, the following formula is used to determine the dosage of the carrier-mediated drug:

Dose=(Predicted clearance)×(Target AUC) Formula 1

where AUC refers to “Area Under the Curve,” which is a measure of the subject's total drug exposure. The AUC can be based on encapsulated and/or released drug.

The target AUC can be based on any desired parameters known in the art and can be determined using routine methods. For example, the target AUC can be selected to achieve a balance between efficacy and side-effects/toxicity.

Accordingly, the invention also provides methods of selecting a dosage of a carrier-mediated drug for a subject based on the predicted clearance rate. In representative embodiments, the method comprises: (a) measuring the activity of phagocytic cells and/or the amount and/or activity of opsonins and/or complement in a biological sample obtained from a subject; (b) predicting the clearance rate of the carrier-mediated drug in the subject based on the measurement of (a); and (c) selecting a dosage of the carrier-mediated drug for the subject from the predicted clearance rate. Optionally, Formula 1 (above) can be used to calculate the dosage from the predicted clearance rate.

Having described the present invention, the same will be explained in greater detail in the following examples, which are included herein for illustration purposes only, and which are not intended to be limiting to the invention.

### Example 1

### PK and PD Variability of Liposomal Anticancer Agents

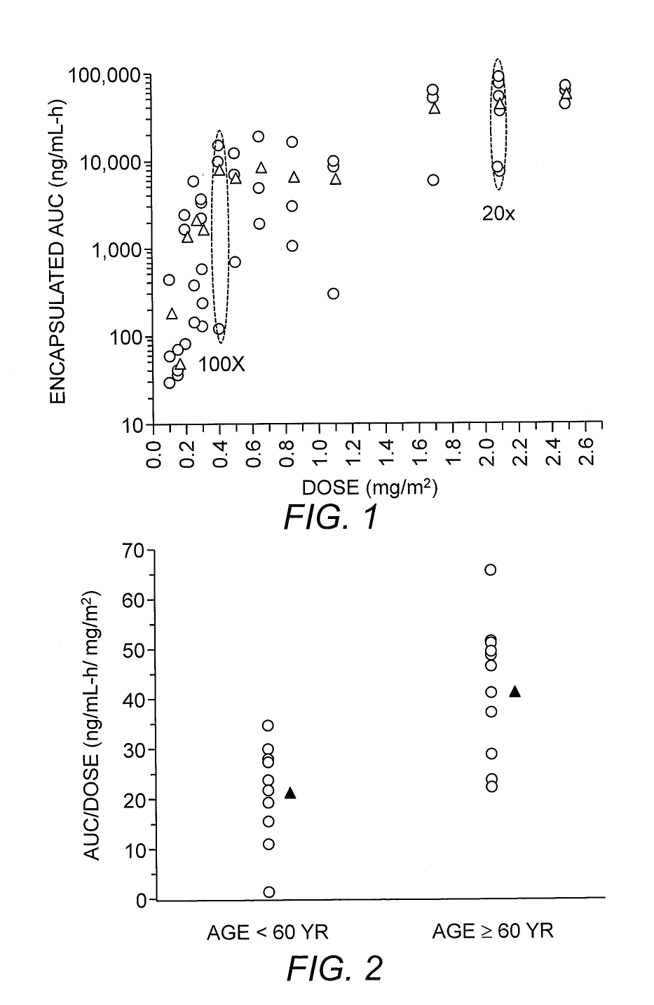

S-CKD602 is a pegylated liposomal formulation of CKD-602, a camptothecin analogue. In a phase I study of S-CKD602 IV every 21 days, we evaluated the PK of liposomal encapsulated and released CKD-602 in plasma. The interpatient variability in the exposure of encapsulated and released CKD-602 ranged from 20- to 100-fold (FIG. 1) and 10-fold, respectively. The interpatient variability in the PK of encapsulated CKD-602 was significantly greater than reported with non-nanoparticle agents administered IV or orally (Gabizon et al., (1994) Cancer Res. 54: 987-992; Amantea et al., (1997) Clin. Pharmacol. Ther. 61: 301-311; La-Beck et al., (2009) J. Clin. Oncol. 27(15S); Zamboni et al., (2009) Clin. Cancer Res. 15:1466-1472; Zamboni et al., (2009) Clin. Pharmacol. Ther. 86: 519-526). We also have reported that the PK variability of S-CKD602 is related to a patient's age, body composition and MO changes (Zamboni et al., (2009) Clin. Cancer Res. 15:1466-1472; Zamboni et al., (2009) Clin. Pharmacol. Ther. 86: 519-526).

Significant interpatient variability in DOXIL® PK and PD has also been observed (Green et al., (2006) Int. J. Nanomedicine 1:229-239; Gabizon et al., (1994) Cancer Res. 54: 987-992; Amantea et al., (1997) Clin. Pharmacol. Ther. 61: 301-311; La-Beck et al., (2009) J. Clin. Oncol. 27(15S)). As a result, we evaluated the effect of age on DOXIL® PK as part of prior phase I studies of DOXIL® in patients with solid tumors (n=23) (Sidone et al., (2007) Evaluation of body surface area (BSA) based dosing, age, and body composition as factors affecting the pharmacokinetic (PK) variability of STEALTH liposomal doxorubicin (Doxil), in Proceedings of AACR-NCI-EORTC, Abstract C107; La-Beck et al., (2009) J. Clin. Oncol. 27(15S)). PK studies were performed on patients receiving DOXIL® at doses of 10 to 60 mg/m² IV every 28 days. The sum total (ST=encapsulated+released) doxorubicin was measured in plasma. Mean±SD DOXIL® ST AUC values normalized by dose in patients that were <60 and ≧60 years of age were 21.1±9.9 and 41.2±13.4 ng/ml·h per mg/m², respectively (P=0.001) (FIG. 2). These data suggest that patients that are ≧60 years of age have an increased DOXIL® exposure (e.g., AUC) and may have increased risk of toxicity as compared with patients <60 years of age. This is significant because the median age at presentation of ovarian cancer is 62 (Bookman et al., (2005) Int. J. Gynecol. Cancer 15 Suppl. 3: 212-220). However, age does not account for all of the PK variability of DOXIL®. The factors associated with this difference are unknown but may involve the RES (Zamboni et al., (2009) Clin. Cancer Res. 15:1466-1472; Zamboni et al., (2009) Clin. Pharmacol. Ther. 86: 519-526).

### Example 2

### PK and PD Relationship Between RES and Carrier-Mediated Chemotherapeutic Drugs

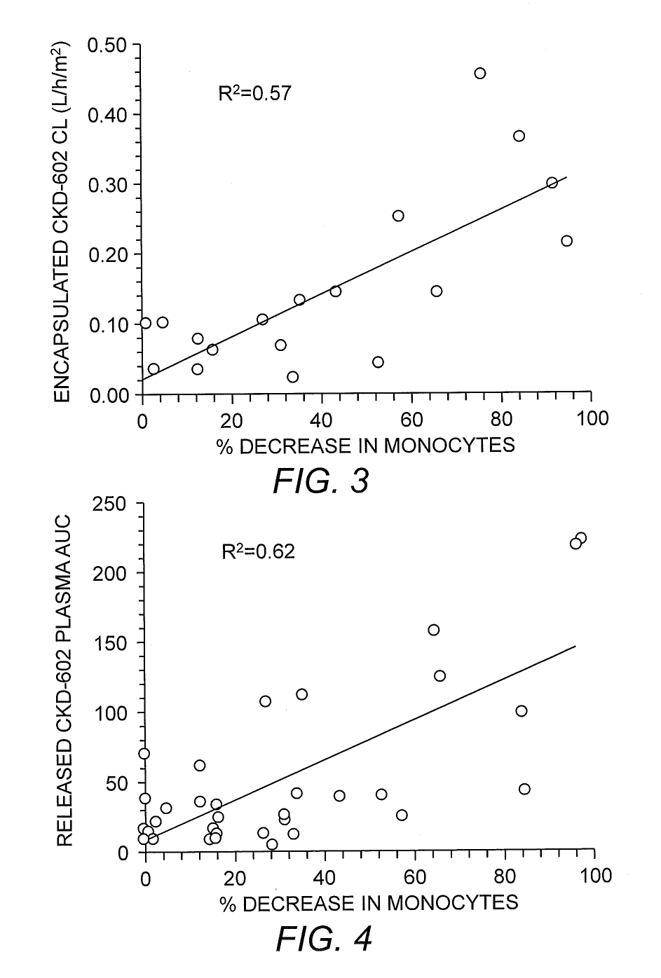

To examine the relationship between S-CKD602 PK and monocytes (MO) in patients, we evaluated the degree of neutropenia and monocytopenia as part of phase I studies of S-CKD602 and non-liposomal CKD-602 (NL-CKD602) in patients with refractory solid tumors. After administration of NL-CKD602, the % decrease at nadir in absolute neutrophil count (ANC) and MO were 86±11% and 87±12%, respectively (P>0.05). For NL-CKD602, the ratio of % decrease in MO to ANC was 1.1±0.4. After administration of S-CKD602, the % decrease at nadir in ANC and MO were 42±30% and 58±34%, respectively (P=0.001). For S-CKD602, the ratio of % decrease in MO to ANC was 2.1±2.0. The relationship between the % decrease in MO at nadir and CL of encapsulated CKD-602 and the release of CKD-602 from S-CKD602 in plasma are presented in FIGS. 3 and 4, respectively. There was no relationship between the pre-treatment MO count in blood and S-CKD602 PK. These data suggest that MO are more sensitive to S-CKD602 as compared with neutrophils, and that the increased sensitivity is related to the liposomal formulation and not the encapsulated drug. The CL of encapsulated drug and release of drug from the liposome in plasma is related to a reduction in MO. This suggests that MO engulf liposomal anticancer agents via their phagocytic function as part of the RES, which subsequently causes drug to be released from the liposome and the ensuing cytotoxicity to the MO. Consistent with the PK-PD interaction between S-CKD602 and MO we also have reported that decreased DOXIL® CL from cycle 1 to 3 is related to a reduction in MO during each subsequent cycle (Gabizon et al., (1994) Cancer Res. 54: 987-992; La-Beck et al., (2009) J. Clin. Oncol. 27(15S)).

### Example 3

### Materials and Methods for PK and PD Studies

### Determining Drug Clearance Rate

Blood samples (5 ml) were obtained one day prior to administration, at the end of in vivo infusion, and at 1 h, 3 h, 24 h, 48 h, 72 h, 96 h, and 168 h after administration of DOXIL®. Liposomal-encapsulated (inactive) and released (active) doxorubicin were separated in plasma via a solid phase separation method. Sum total (encapsulated and released), encapsulated, and released doxorubicin concentrations were evaluated in plasma. Sum total and encapsulated fractions were extracted from plasma using liquid-liquid extraction with protein precipitation. Released fractions were directly eluted from the solid phase separation matrix. Doxorubicin concentrations were measured using HPLC-fluorescence. The lower limit of quantitation for both plasma sum total and encapsulated doxorubicin assays was 10 ng/mL, the upper limit of quantitation was 30,000 ng/mL. The lower and upper limits of quantitation for the plasma released doxorubicin assay were 50 ng/mL and 3,000 ng/mL, respectively. Pharmacokinetic parameters were calculated via noncompartmental modeling using WinNonLin 5.2 (Pharsight).

### Phagocytic Cell Activity

Blood samples (10 mL) were obtained prior to administration of DOXIL®, and at 48 h, and 96 h after DOXIL® administration. Samples were processed within 2 hrs of sample collection to determine the number and function of monocytes/dendritic cells via flow cytometry using the Dako Cyan flow cytometer and analyzed with Summit 5.2 software.

Granulocytes, T-lymphocytes, and B-lymphocytes were identified and quantified using fluorochrome labeled antibodies against CD15, CD3, and CD19, respectively. Monocytes were identified and quantified (absolute number and/or percentage) using fluorochrome labeled monoclonal antibodies against CD14 and CD16 which enables evaluation of two monocyte subpopulations.

Two dendritic cell populations, myeloid dendritic cells and lymphoid dendritic cells, were identified and quantified using four color flow cytometry. A cocktail of fluorochrome labeled monoclonal antibodies against CD3, CD16, CD19, CD14, CD20, CD34, CD56, and HLA-DR enables identification of dendritic cells that are characterized by the absence of these CD lineage markers (“lineage negativity”) and high expression of HLA-DR. In addition, antibodies against CD11c and CD123 distinguish between myeloid dendritic cells (high expression of CD11c but low expression of CD123) and lymphoid dendritic cells (low expression of CD11c and high expression of CD123).

PHAGOTEST® (Orpegen Pharma, Heidelberg, Germany) was used to assess the different phagocytic capacities of the blood cells identified above. This assay utilizes fluorescein labeled Escherichia coli particles, which were detected by a 488 nm laser at ˜530 nm (FITC). Quantification of the number of particles phagocytized by each cell was enabled by using the flow cytometer.

PHAGOBURST® (Orpegen Pharma, Heidelberg, Germany) was used to assess the respiratory burst activity of blood cells identified above. Upon cell stimulation, a series of intracellular pathways were activated which generated reactive oxygen species as a by-product. These reactive oxygen species reacted with dihydrorhodamine (which is not fluorescent) resulting in the formation of rhodamine, which is fluorescent, and was detected by the 488 nm laser at ˜530 nm (FITC). The respiratory burst activity of cells was quantified using the Dako Cyan flow cytometer.

Cellular uptake of DOXIL® (liposome encapsulated) and doxorubicin (non-liposomal) was measured by flow cytometry due to the intrinsic fluorescence of DOXIL® and doxorubicin in the far red spectrum (excitation by 488 nm laser and emission fluorescence at 575 nm).

In some experiments, blood samples were pre-incubated for 1 hr at 37° C. and 5% CO₂, with and without addition of DOXIL® at 10 mcg/mL. Samples were subsequently processed as described above to identify and quantify granulocytes, T-lymphocytes, B-lymphocytes, monocytes, and dendritic cells. Samples were also processed to evaluate DOXIL® cellular uptake, respiratory burst activity, and phagocytic activity.

### Example 4

### Phenotypic Probes for DOXIL® PK and PD

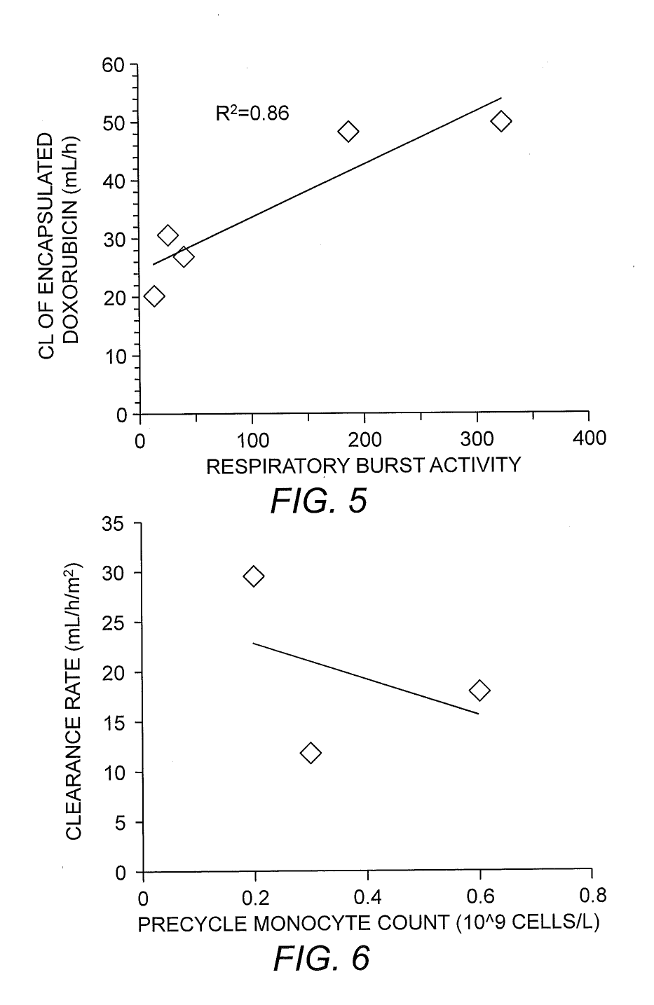

The function of MO and dendritic cells (DC) in blood as phenotypic probes for DOXIL® PK and PD was evaluated in women with recurrent ovarian cancer as described in Example 3. Our results on the relationship between in vivo (no DOXIL® added ex vivo) MO oxidative burst activity in blood prior to the administration of DOXIL® alone (n=3) and in combination with carboplatin (n=2) and CL of encapsulated doxorubicin in 5 patients is presented in FIG. 5. There was no difference in DOXIL® CL with and without carboplatin. There was a significant relationship between MO oxidative burst activity and DOXIL® CL (R²=0.83). There was no relationship between pre-treatment MO count and DOXIL® CL (R²=0.17).

These data demonstrate that patients with higher MO activity in the blood have a higher DOXIL® CL. In theory, the relationship between MO activity in blood and DOXIL® CL may be due to MO in blood being a major elimination pathway for DOXIL® and potentially other nanoparticle agents and/or that the MO activity in the blood is a surrogate marker for the overall MPS activity in the body. In either case, our data indicate that MO activity in blood can used to predict DOXIL® CL and individualize therapy.

### Example 5

### Correlation of In Vivo Phagocytic Activity with Measured Drug Clearance Rate

A correlation was determined between the drug clearance rate (CL) of DOXIL® in monocytes and the phagocytic activity of monocytes. The in vivo monocyte phagocytic activity in whole blood was determined using the PHAGOTEST® assay (Orpegen Pharma, San Diego, Calif.) as described in Example 3. DOXIL® CL was also determined as described in Example 3.

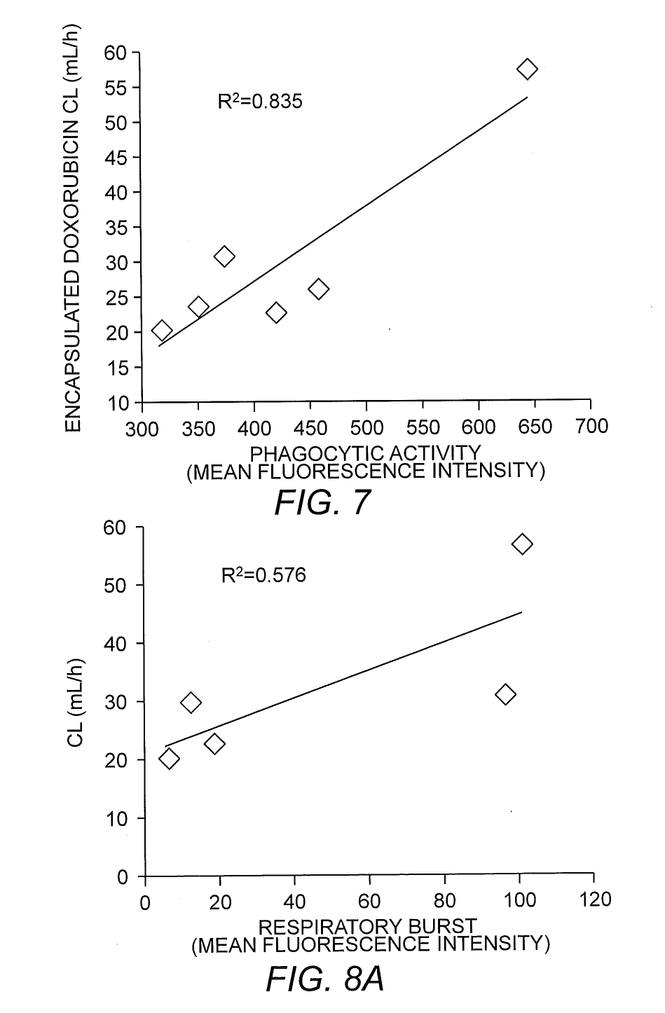

The relationship between monocyte count determined from a complete blood count (CBC) prior to DOXIL® administration and DOXIL® CL is presented in FIG. 6. The DOXIL® CL represents the CL of the liposomal encapsulated doxorubicin. We previously reported that the overall pharmacokinetic and pharmacodynamic disposition of nanoparticle and liposomal agents are dictated by how the carrier is handled by the reticuloendothelial cell system (RES) system. The relationship between in vivo monocyte phagocytic activity in blood prior to DOXIL® administration and DOXIL® CL is presented in FIG. 7. There was a significant relationship between monocyte phagocytic activity and DOXIL® CL (R²=0.97), but not between precycle monocyte count and DOXIL® CL (R²=0.17).

These results demonstrate a significant relationship between in vivo probes of monocyte phagocytic activity and DOXIL® clearance. These results further indicate that patients with a higher phagocytic activity have a higher rate of clearance of DOXIL®, and that in vivo monocyte phagocytic activity can be used to predict the clearance of DOXIL® and other carrier-mediated drugs and individualize therapy.

This study can be carried out using other phagocytic cells (e.g., dendritic cells and/or macrophages) including subpopulations thereof.

### Example 6

### Correlation of Respiratory Burst Activity with Measured Drug Clearance Rate

A correlation was demonstrated between the drug clearance rate of DOXIL® and the fMLP-induced respiratory burst activity of monocytes. The in vivo monocyte respiratory burst activity in whole blood using fMLP was measured using the PHAGOBURST® kit (Orpegen Pharma, San Diego, Calif.) as described in Example 3. DOXIL® CL was also determined as described in Example 3.

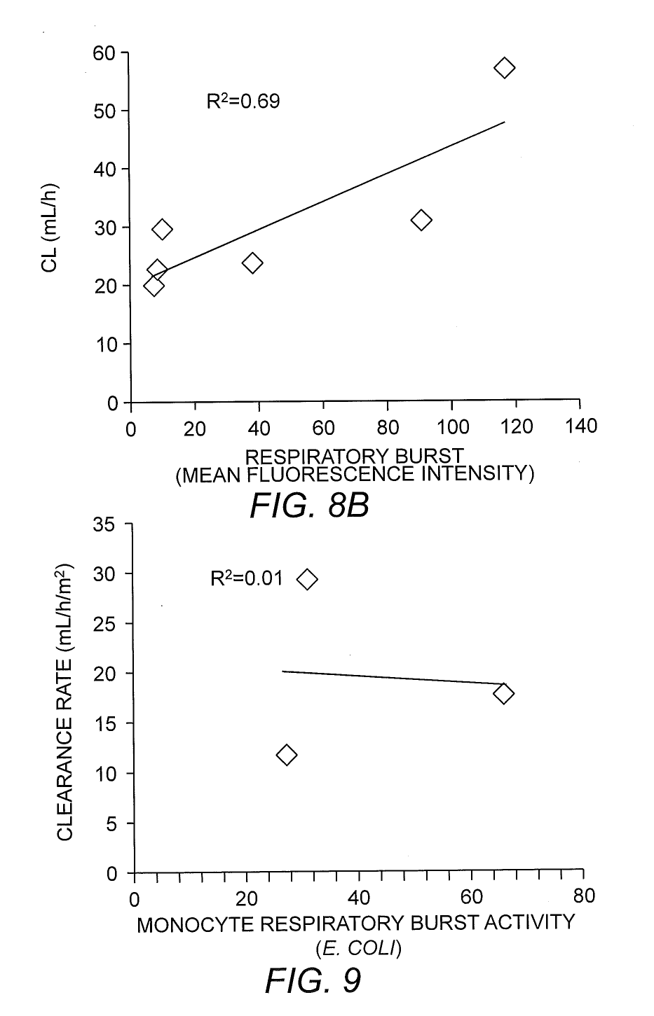

The relationship between in vivo monocyte respiratory burst activity in blood after fMLP on day 1 prior to DOXIL® administration and DOXIL® CL is presented in FIG. 8A. There was a significant relationship between monocyte respiratory burst activity by fMLP and DOXIL® CL (R²=0.576).

The relationship between in vivo monocyte respiratory burst activity in blood without the addition of any stimulants (e.g., PMA, fMLP) on day 1 prior to DOXIL® administration and DOXIL® CL is presented in FIG. 8B. There was a significant relationship between monocyte respiratory burst activity and DOXIL® CL (R²=0.69).

Our data demonstrate a significant relationship between in vivo probes of monocyte respiratory burst activity and DOXIL® clearance. These results indicate that patients with a higher monocyte respiratory burst activity have a higher rate of clearance of carrier-mediated drugs. In vivo respiratory burst activity can be used to predict the clearance of DOXIL® and other carrier-mediated drugs and individualize therapy.

This study can be carried out using other phagocytic cells (e.g., dendritic cells and/or macrophages) including subpopulations thereof.

### Example 7

### The Relationship Between In Vivo Respiratory Burst Activity Using E. coli or PMA and Measured Drug Clearance

In this study, we found no correlation between the drug clearance rate of DOXIL® in monocytes and the E. coli- or PMA-induced respiratory burst activities of monocytes. The in vivo monocyte respiratory burst activity in whole blood using E. coli or PMA was measured using the PHAGOBURST® kit (Orpegen Pharma, San Diego, Calif.) as described in Example 3.

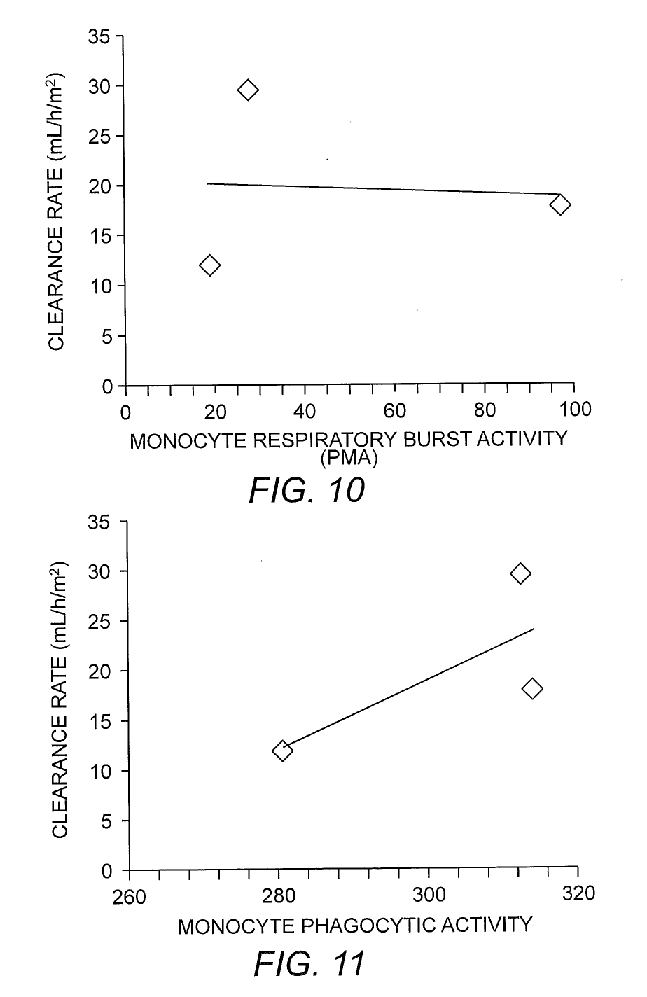

The relationship between in vivo monocyte respiratory burst activity using E. coli and PMA on day 1 prior to DOXIL® administration and DOXIL® CL is presented in FIGS. 9 and 10, respectively. There was no relationship between in vivo monocyte respiratory burst activity in blood using E. coli (R²=0.01) or PMA (R²=0.01) on day 1 prior to DOXIL® administration and DOXIL® CL. In contrast, the in vivo phagocytic activity and respiratory burst activity using fMLP are predictive of monocyte function related to DOXIL® CL (R²=0.62; Example 6); however, respiratory burst activity using E. coli and PMA were not.

### Example 8

### Correlations of Measured Drug Clearance Rate with Phagocytic Activity or Respiratory Burst Activity in a Blood Sample Following Ex Vivo Addition of Carrier-Mediated Drug

Correlations were demonstrated between the drug clearance rate of DOXIL® in monocytes and the phagocytic and respiratory burst activity of monocytes.

Methods:

Phagocytic activity with the ex vivo addition of carrier-mediated drug was determined as described in Example 3. DOXIL® CL was also determined as described in Example 3.

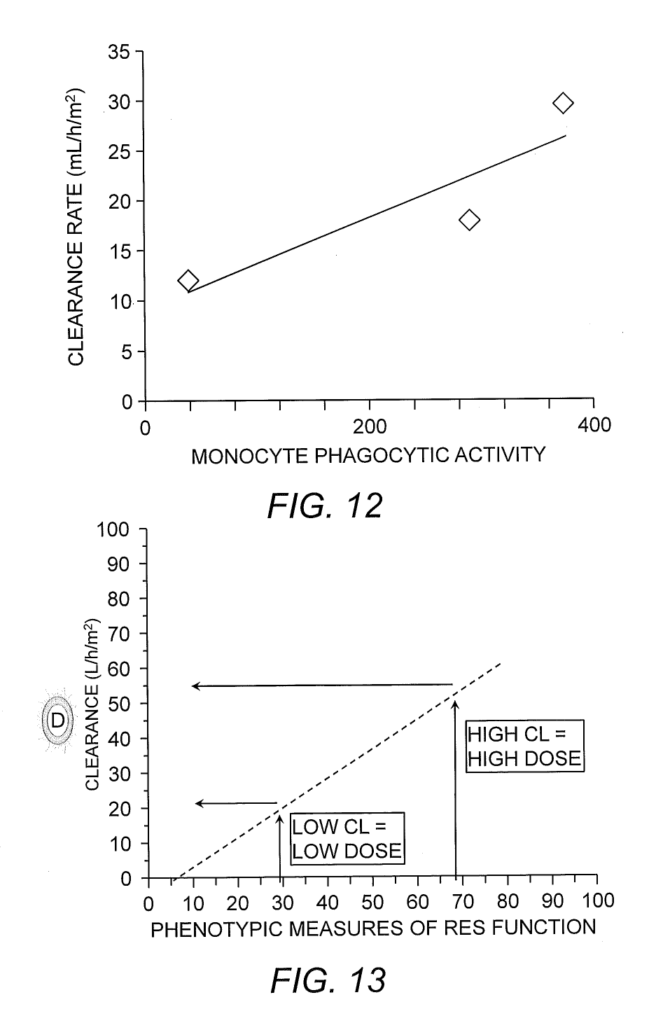

The relationship between monocyte phagocytic activity in blood prior to DOXIL® administration without and with the ex vivo addition of DOXIL® and DOXIL® CL is presented in FIGS. 11 and 12, respectively. There was a significant relationship between monocyte phagocytic activity and DOXIL® CL alone (R²=0.53) and with the ex vivo addition of DOXIL® (R²=0.80); however, the ex vivo addition of DOXIL® significantly improved the relationship between the probe and DOXIL®, CL.

Monocyte respiratory burst activity was also measured (using fMLP, E, coli or PMA as stimulants) with and without the ex vivo addition of DOXIL® and the relationship with DOXIL® CL was determined. Results are shown in Table 2.

This study can be carried out with other phagocytic cells (e.g., macrophages and/or dendritic cells) and subtypes thereof.

The relationships between monocyte phenotypic probes with and without the ex vivo addition of DOXIL® are summarized in Table 2.

TABLE 2
A summary of the relationships between monocyte phenotypic probes
with and without the ex vivo addition of DOXIL ®

| | | Relationship between | |
|---|---|---|---|
| | | Probe and DOXIL ®CL | |
| | | Without | With |
| | | Ex Vivo | Ex Vivo |
| Cells | Phenotypic Probe | DOXIL ® (R²) | DOXIL ® (R²) |
| Monocytes | Phagocytic Activity | 0.53 | 0.80 |
| Monocytes | fMLP Respiratory Burst | 0.89 | 0.99 |
| | Activity | | |
| Monocytes | E. coli Respiratory | 0.83 | 0.94 |
| | Burst Activity | | |
| Monocytes | PMA Respiratory Burst | 0.56 | 0.81 |
| | Activity | | |

The ex vivo addition of DOXIL® to the phenotypic probes improves the predictive relationship between the probe and DOXIL® CL. Unexpectedly, there was no relationship (R²=0.01) between the concentration of DOXIL® in monocytes after the ex vivo addition of DOXIL® to blood and the clearance of DOXIL® (data not shown). Thus, the effects of the carrier-mediated drug on the function and/or pharmacodynamics of the monocyte predict the clearance of the carrier-mediated drug and not the amount of drug that is taken up by the monocyte.

### Example 9

### Selection of Individualized DOXIL® Dose Based on Phenotypic Probes

Precycle respiratory burst activity or phagocytic activity of phagocytic cells in whole blood can be used to predict DOXIL® CL and select an individualized dosage of DOXIL® on that basis.

Blood samples are obtained from a population of patients with recurrent, platinum-resistant ovarian cancer on day 7 pre-cycle. Using the PHAGOTEST® and PHAGOBURST® kits (Orpegen Pharma, San Diego, Calif.), phagocytic activity and/or respiratory burst activity of phagocytic cells (e.g., MO and/or DC) in the blood sample is measured as described in Example 3. DOXIL® CL is also determined as described in Example 3. The results are used to generate a standard curve (see, e.g., FIG. 13).

A blood sample is obtained from an individual patient with recurrent, platinum-resistant ovarian cancer on day 7 pre-cycle, and phagocytic activity and/or respiratory burst activity of phagocytic cells (e.g., MO and/or DC) in the blood sample is determined. The predicted DOXIL® CL is then obtained from the standard curve (FIG. 13). Dosage varies linearly with DOXIL® CL and can be determined with Formula 1.

Dose=(Predicted clearance)×(Target AUC of encapsulated and/or released drug) Formula 1:

The Target AUC is selected to optimize the balance between therapeutic efficacy and toxicity and other side effects.

In general, as shown in FIG. 13, a relatively low DOXIL® CL translates to a relatively low dose of DOXIL®, whereas a relatively high DOXIL® CL corresponds to a higher dosage being administered.

The calculated dosage is administered to the patient. The phenotypic probe can be used to recalculate the appropriate DOXIL® dosage over the course of the chemotherapy regimen. For example, the dosage can be recalculated before every cycle, every other cycle, every three cycles, etc.

### Example 10

### Phase I Clinical Trial for the Evaluation of the Function of MO/DC as Phenotypic Probes for DOXIL®PK and PD in Recurrent Ovarian Cancer Clinical Study Design and Patients

Clinically significant PK and PD variability of DOXIL® exists among patients. Furthermore, the effective DOXIL® dose for a given patient is only slightly lower than the toxic dose. Hence, accurate prediction and calibration of the dose to administer to each patient is important. The ability to measure the phagocytic function or activity of MO and DC in blood samples can be used to predict the DOXIL® PK and PD (efficacy and toxicity) in patients.

This study evaluating phenotypic measures of the RES and DOXIL® PK and PD is performed at the University of North Carolina (UNC) Clinical Trials Research Center (CTRC) and UNC Gynecologic Oncology Clinic in 50 women ≧18 years of age receiving DOXIL® alone or in combination with carboplatin as treatment for recurrent ovarian cancer. DOXIL® is administered IV over 1 to 2 h at 30 or 40 mg/m² every 28 days alone or in combination with carboplatin IV to achieve an AUC of 5 mg/mL·min. The phenotyping and DOXIL® PK and PD studies are performed for cycle 1 only. DOXIL® is administered on day 1. Serial ex vivo and in vivo phenotypic probe studies of MO/DC number and function in blood of patients are performed before and after DOXIL® administration. Plasma and PBMC PK studies are performed on day 1 to 28.

### Number and Function of MO/DC.

Blood samples (10 mL) are obtained prior to administration of DOXIL® (on day −7 to −1 and day 1), and at 48 h, and 96 h after DOXIL® administration, and prior to the dose on cycle 2. Blood is processed to determine the number and function of MO/DC via flow cytometry (FCM) in the UNC Flow Cytometry Core Lab in collaboration with Dr. Lay. Blood samples are processed within 2 h of sample collection. Antibodies are obtained from BD Biosciences, San Jose, Calif. and flow cytometric analyses are performed using the Dako Cyan FCM with Flowjo 7.6.1 software.

MO and DC cells are identified and quantified (absolute number and/or percentage) in two separate flow cytometric analyses (Autissier et al., (2010) Cytometry (Part A). 77:410-419). Fluorochrome labeled monoclonal antibodies against CD14 and CD16 are used to identify and quantify MO subpopulations using two color FCM (Mittag et al., (2005) Cytometry (Part A) 65:103-115). Two DC populations, myeloid DC and lymphoid DC, are identified and quantified using four color FCM. A lineage cocktail of fluorochrome labeled monoclonal antibodies enables identification of DC subpopulations (Autissier et al., (2010) Cytometry (Part A). 77:410-419).

PHAGOBURST® (Orpegen Pharma, Heidelberg, Germany) is used to assess the respiratory burst activity of MO and DC. Upon cell stimulation with fMLP, PMA, or Escherichia coli, a series of intracellular pathways are activated that generate reactive oxygen species as a by-product. Reactive oxygen species react with dihydrorhodamine (not fluorescent) resulting in the formation of fluorescent-rhodamine which is detected by a 488 nm laser at ˜530 nm (FITC). Respiratory burst activity of cells is quantified using the Dako Cyan FCM.

In order to assess the different phagocytic capacity of MO and DC, blood samples are analyzed using the PHAGOTEST® (Orpegen Pharma, Heidelberg, Germany). This assay utilizes fluorescein labeled E. coli particles which are detected by a 488 nm laser at ˜530 nm (FITC). Quantification of the number of particles phagocytized by each cell will be performed by FCM.

### MO/DC Changes in Response to Ex Vivo DOXIL® Exposure.

Blood samples are obtained prior to administration of DOXIL® on day −7 to −1. FCM studies of MO/DC function (PHAGOBURST®; PHAGOTEST®) are performed with and without ex vivo incubation with DOXIL® (10 μg/mL×1 h) in the UNC Flow Cytometry Core Lab. Samples are also assessed for changes in cell viability using standard methods (Schonn et al., (2010) Apoptosis 15:162-172).

### Plasma and PBMC PK Studies.

Blood samples (5 mL) are obtained prior to administration, at the end of infusion (EOI), and at 1 h, 3 h, 6 h, 24 h, 48 h, 72 h, 96 h, 168 h and day 28 after administration of DOXIL®. Blood is processed to measure encapsulated (i.e., DOXIL®), released and sum total (encapsulated+released) doxorubicin in plasma (Zamboni et al., (2009) Clin. Cancer Res. 15:1466-1472; Zamboni et al., (2007) Clin. Cancer Res. 13: 7217-7223). Blood is also processed to measure sum total doxorubicin in PBMC (ter Heine et al., (2009) J. Chromatogr. B Analyt. Technol. Biomed. Life Sci. 877: 575-580; Devalapally et al., (2009) Use of PBMC Partitioning to predict RES-uptake of nanoemulsion in a colon cancer xenograft, in nanotech Conference & Expo, May 3-7, 2009, Houston, Tex.). The concentration of doxorubicin in these samples is determined using the HPLC-FL assay described in Example 3.

### PK and PD Analyses.

Compartmental and non-compartmental PK analyses are performed for encapsulated, released and sum total doxorubicin in plasma and for sum total doxorubicin in PBMC. WinNonLin software is used to calculate the clearance (CL), volume of distribution (Vd), half-life (t½), and area under the concentration versus time curve (AUC) of each form (Sheiner et al. (1980) J. Pharmacokinet. Biopharm 8: 553-571; Gabrielsson et al., (2000) Pharmacokinetic and pharmacodynamic data analysis: concepts and applications. 3rd ed., Taylor & Francis, p. 311; Beal et al., NONMEM users Guide, 1989-2006; Rowland et al., (1989) Clinical pharmacokinetics: concepts and applications, Philadelphia, Lea & Febiger). Population PK and covariate analyses are performed using NONMEM (Beal et al., NONMEM users Guide, 1989-2006; Mandema et al., (1992) J. Pharmacokinet. Biopharm. 20: 511-528).

### Statistical Design and Sample Size.

A major aim of this study is to evaluate the ability of cellular phenotypic probes to predict DOXIL® PK and PD in patients with recurrent ovarian cancer. A baseline blood sample is used to measure various probes [e.g., MO respiratory burst activity (RBA)], while CL of encapsulated doxorubicin is estimated from PK data in each patient. To evaluate a given probe (e.g., RBA) as a predictor of CL, the statistical approach is to test the null hypothesis that the correlation between CL and RBA is 0.5 (R²=0.25) against the alternative that it is 0.75 (R²=0.65) or greater, reflecting a clinically meaningful predictive ability. The planned sample size of n=50 provides power 0.89. A Bonferroni adjustment for testing 5 probes, with overall level 0.05, gives power 0.71.

### Statistical Analysis Plan.

Hypothesis testing is performed as described above. Further, XY plots are used to graphically explore relationships among variables. The non-parametric Spearman correlation is used to quantify the strength of increasing (or decreasing) trends that are not necessarily linear (Hollander et al., (1999) Nonparametric statistical methods. 2^{nd} ed. Wiley series in probability and statistics. Texts and references section. New York: Wiley. P. 394).

### Example 11

### Selection of a Carrier-Mediated Agent Based on Desired Level of Interaction with the RES System

The phenotypic probes can be used to evaluate the interaction between the RES and a carrier-mediated agent (e.g., a carrier-mediated drug), and to pick a carrier-mediated agent having a desired or target level of interaction (e.g., stimulation) of the RES.

Human blood samples are incubated ex vivo with one of eight different carrier-mediated agents. These can be variations on a single formulation (shown as different nanoparticle formulations) or can be eight different carriers that can each deliver the same agent. The 8 carrier-mediated agents are characterized using one or more phenotypic probes to measure the activity of phagocytic cells (for example, MO, macrophage and/or DC), e.g., phagocytic activity, respiratory burst activity, chemotaxis, receptor binding, generation of superoxide, generation of nitric oxide, presentation of one or more antigens at the cell surface, or any combination thereof. The results can be plotted (FIG. 14).

As shown in the left half of FIG. 14, a carrier-mediated agent can be selected with low effects on the MPS/RES. This profile may be desirable, for example, for a carrier-mediated chemotherapeutic drug, e.g., so that there is less interaction between the chemotherapeutic drug and the cells of the RES, which may reduce toxicity to the RES and immune system. In other instances, however, it may be advantageous to select a carrier-mediated agent with a relatively strong effect on the MPS/RES (e.g., a carrier-mediated drug to treat an immune system disorder), as shown in the right half of FIG. 14, e.g., a drug that interacts strongly and is targeted to the cells of the RES/immune system.

The foregoing is illustrative of the present invention, and is not to be construed as limiting thereof. The invention is defined by the following claims, with equivalents of the claims to be included herein.

1. A method of predicting the clearance rate of a carrier-mediated agent in a subject, the method comprising:
a) measuring the activity of phagocytic cells in a biological sample obtained from the subject; and
b) predicting the clearance rate of the carrier-mediated agent in the subject based on the activity of the phagocytic cells in the biological sample.

2. A method of predicting the release of an agent from a carrier-mediated agent, the method comprising:
a) measuring the activity of phagocytic cells in a biological sample obtained from the subject; and
b) predicting the release of the agent from the carrier-mediated agent in the subject based on the activity of the phagocytic cells in the biological sample.

3. The method of claim 1, wherein the carrier-mediated agent is a carrier-mediated drug.

4. The method of claim 3, wherein the carrier-mediated drug is pegylated liposomal encapsulated doxorubicin, liposomal daunorubicin, liposomal cytarabine, paclitaxel albumin-bound particles, amphotericin B liposome, amphotericin B lipid complex or pegylated liposomal CKD-602.

5. The method of claim 1, wherein the biological sample is contacted with the carrier-mediated agent prior to measuring the activity of phagocytic cells in the biological sample.

6. The method of claim 1, wherein the biological sample is contacted with the carrier prior to measuring the activity of phagocytic cells in the biological sample.

7. The method of claim 1, wherein the actual clearance rate of the carrier-mediated agent in the subject is determined and compared with the predicted clearance rate.

8. The method of claim 2, wherein the actual release of the agent from the carrier-mediated agent in the subject is determined and compared with the predicted release.

9. The method of claim 1, wherein predicting the clearance rate of the carrier-mediated agent and/or predicting the release of the agent from the carrier-mediated agent comprises comparing the activity of the phagocytic cells within the biological sample to a reference value.

10. The method of claim 1, wherein the method further comprises obtaining the biological sample from the subject.

11. The method of claim 1, wherein the sample is a blood sample, plasma sample, serum sample, ascites sample, or any combination of the foregoing.

12. The method of claim 1, wherein the subject is a human subject.

13. The method of claim 1, wherein the subject is receiving or will receive chemotherapy.

14. The method of claim 13, wherein the method is carried out prior to two or more cycles of chemotherapy.

15. The method of claim 13, wherein the method is carried out prior to every cycle of chemotherapy.

16. The method of claim 1, wherein the carrier-mediated agent comprises a liposome, a nanoparticle, a conjugate and/or a polymer.

17. The method of claim 16, wherein the carrier-mediated agent comprises a stabilized liposome, a non-stabilized liposome, a nanosphere, a microsphere, a dendrimer, a quantum dot, a gold nanoshell, a nanocrystal, colloidal gold, a nanoemulsion, an antibody, a viral vector, a virus-like particle, a carbon nanotube, a gold nanoparticle, a silver nanoparticle, a silica nanoparticle, a conjugate, a polymer, or any combination of the foregoing.

18. The method of claim 1, wherein the activity of phagocytic cells is measured by evaluating phagocytosis, respiratory burst activity, chemotaxis, receptor binding, generation of superoxide, generation of nitric oxide, presentation of one or more antigens at the cell surface, or any combination of the foregoing.

19. The method of claim 1, wherein the phagocytic cells comprise monocytes, macrophages, dendritic cells, granulocytes, mast cells, lymphocytes, or any combination of the foregoing.

20. The method of claim 19, wherein the phagocytic cells comprise monocytes, macrophages, dendritic cells or any combination of the foregoing.

21. The method of claim 1, wherein the method further comprises determining the amount and/or activity of opsonins in the biological sample.

22. The method of claim 1, wherein the method further comprises determining the amount and/or activity of complement in the biological sample.

23. The method of claim 1, wherein the carrier-mediated agent comprises a detectable label.

24. A method of selecting a dosage of a carrier-mediated drug for a subject, the method comprising:
a) measuring the activity of phagocytic cells in a biological sample obtained from the subject;
b) predicting the clearance rate of the carrier-mediated drug in the subject based on the activity of the phagocytic cells in the biological sample; and
c) selecting a dosage of the carrier-mediated drug for the subject from the predicted clearance rate.

25. The method of claim 24, wherein the method further comprises administering the dosage of the carrier-mediated drug to the subject.

26. The method of claim 24, wherein the carrier-mediated drug is pegylated liposomal encapsulated doxorubicin.

27. The method of claim 24, wherein the biological sample is contacted with the carrier-mediated drug prior to measuring the activity of phagocytic cells in the biological sample.

28. A method of predicting the activity of the reticuloendothelial cell system (RES) in a subject, the method comprising:
a) measuring the activity of phagocytic cells in a biological sample obtained from the subject; and
b) predicting the activity of the RES in the subject based on the activity of the phagocytic cells in the biological sample.

29. The method of claim 28, wherein the biological sample is contacted with a carrier-mediated agent prior to measuring the activity of phagocytic cells in the biological sample.

30. A method of identifying a carrier-mediated agent having a desired effect on and/or interaction with the RES, the method comprising:
a) measuring the activity of phagocytic cells in a biological sample obtained from a subject;
b) predicting the effect of the carrier-mediated agent on the RES and/or the level of interaction of the carrier-mediated agent with the RES in the subject based on the activity of the phagocytic cells in the biological sample; and
c) identifying a carrier-mediated agent with a predicted effect on the RES and/or level of RES interaction in a target range based on the prediction of (b).

31. The method of claim 30, wherein the method is carried out with two or more carrier-mediated agents.

32. The method of claim 30, wherein the biological sample is contacted with the carrier-mediated agent prior to measuring the activity of phagocytic cells in the biological sample.
